(19) United States
(12) Patent Application Publication
(10) Pub. No.: US 2025/0281679 A1
(43) Pub. Date: Sep. 11, 2025

(54) BIOMIMETIC COATING FOR ENDOVASCULAR STENT

(71) Applicants: INSERM (Institut National de la Santé et de la Recherche Médicale), PARIS (FR); UNIVERSITÉ PARIS CITÉ, PARIS 6 (FR); UNIVERSITE PARIS XIII PARIS-NORD, VILLETANEUSE (FR); ALCHIMEDICS, PARIS (FR)

(72) Inventors: Giuseppina CALIGIURI, PARIS (FR); Antonino NICOLETTI, PARIS (FR); Charles SKARBEK, PARIS (FR); Isabelle LEFEVRE, PARIS (FR); Christophe BUREAU, PARIS (FR)

(73) Assignees: INSERM (Institut National de la Santé et de la Recherche Médicale), PARIS (FR); UNIVERSITÉ PARIS CITÉ, PARIS 6 (FR); UNIVERSITE PARIS XIII PARIS-NORD, VILLETANEUSE (FR); ALCHIMEDICS, PARIS (FR)

(21) Appl. No.: 18/715,588

(22) PCT Filed: Dec. 2, 2022
(86) PCT No.: PCT/IB2022/000684

§ 371 Date: May 31, 2024

(30) Foreign Application Priority Data
Dec. 3, 2021 (WO) IB2021/000860

(51) Int. Cl. A61L 31/08

(52) U.S. Cl. CPC A61L 31/08; A61L 2400/18

(57) ABSTRACT

Inventors have synthesized peptides allowing an engagement of intact CD31 molecules on all the healthy endothelial cells and resting blood platelets and leukocytes that can enter in contact with an implanted device. Those cells can therefore receive the “leave-me-alone” signal delivered by the trans-homophilic engagement of CD31, which is essential to maintain the homeostasis in the circulation and vascularized tissues. Thrombotic or life-threatening occurrence of hemorrhagic or thromboembolic complications have impaired the use of endovascular devices. The devices bearing the mimicking peptides of the present invention are rapidly integrated, because they are perceived by blood platelets and leukocytes as a healthy endothelium, a “self’ component. Furthermore, their ability to be rapidly endothelialized with a physiologic endothelial cell phenotype also limits platelet and leukocyte activation at the site of device implantation in the long-term. Accordingly, the present invention relates to peptides mimicking the trans-homophilic CD31-CD31 domain 1 and 2 intercellular interaction.

### REFERENCE TO ELECTRONIC SEQUENCE LISTING

The application contains a Sequence Listing which has been submitted electronically in .XML format and is hereby incorporated by reference in its entirety. Said .XML copy, created on Nov. 7, 2024, is named “B384967US D43362_Sequence listing_2024-11-07.xml” and is 84,543 bytes in size. The sequence listing contained in this .XML file is part of the specification and is hereby incorporated by reference herein in its entirety.

### FIELD OF THE INVENTION

The present invention finds application in the medical field and in particular to improve the integration and performance of implantable medical devices.

### BACKGROUND OF THE INVENTION

Strategies aimed at improving the biocompatibility of medical devices are needed to prevent the reaction of living tissues to the implantation of medical devices, potentially driving unexpected risk for the patients.

A “foreign body response” is caused by the injury of the tissue at the site of implantation and initiated by the contact of the non-biocompatible material with the blood.

The adherence and activation of platelets followed by the recruitment of inflammatory cells is followed by an aberrant wound healing sequence characterized by a chronic inflammation and the formation of granulomatous tissue.

Upon the occurrence of an injury, the interruption of the endothelial barrier allows the activation of platelets, to seal off the damage, and infiltration of leukocytes to clear the debris and prepare the site for tissue regeneration.

Medical devices however cannot be cleared.

As a consequence, in the absence of a prompt endothelialization, there is a strong activation of leucocytes and platelets at the site of implantation.

The excessive platelet adhesion and aggregation can cause the obstruction of the blood flow (ischemia) whereas the proteases released by activated platelets and leukocytes can lead to the rupture of the vascular wall (hemorrhage).

One of such condition is triggered by the presence of heart valves and vascular devices, which are seen as foreign elements to the body.

That has and is hampering the use of valvular and vascular devices for the prevention and treatment of severe cardiovascular conditions.

CD31 is a transmembrane glycoprotein expressed constitutively and exclusively on platelets, leukocytes, and endothelial cells (EC). Under healthy conditions, the trans-homophilic CD31-CD31 interaction allows the recognition of the “self”′ by endothelial cells, platelets, and leukocytes preventing their inappropriate activation.

The favorable issue of blood-contacting implantable devices can be achieved by treatments rendering their surface “biocompatible” to allow the rapid formation of a functional endothelial layer on them and ensuring a proper integration of the biomaterial.

The abundant expression of CD31 by the healthy endothelium exerts an essential role for the maintenance of the homeostasis in the circulation and vascularized tissues.

Cortese et al (Stroke, February 2021) discloses that the immobilization of a CD31-mimetic peptide P8RI (kwpalfvr) reduces the blood element reaction, increases the adhesion of endothelial cells in vitro and enhances the integration of endovascular devices in vivo.

Diaz-Rodriguez et al (EHJ, 2021) discloses that the soluble peptide referred to as P8RI acts like a CD31 agonist; therefore, it has been studied the effect of CD31-mimetic metal stent coating on the in vitro adherence of endothelial cells and blood elements and the in vivo endothelial strut coverage and foreign body response-driven neointimal growth.

CD31 is a type I transmembrane glycoprotein composed of 6 extracellular Ig-like domains, numbered starting from the membrane distal N-terminus, a short transmembrane fragment, and a cytoplasmic tail.

The engagement of CD31 depends upon a trans-homophilic interaction between domains 1 and 2 of the molecules expressed by a first cell A and the same domains expressed by the interacting cell B.

As per the interacting domains 1 and 2 (referred to respectively as “IgL1” and “IgL2”), more specifically, they adopt the classical Ig domain conformation comprised of two layers of β-sheets possessing antiparallel β-strands anchored by a pair of cysteines forming a disulfide bond.

Upon trans-homophilic interaction between the CD31 molecules of two interacting cells, the CD31 dimer interface includes hydrophobic and hydrophilic interactions.

The two IgL1-2 fragments of the trans-homophilic interacting CD31 molecules are packed to each other in a face-to-face antiparallel pattern with the side face of one β sheet (IgL1 interacts with IgL2 from the opposite monomer).

In the crystal structure there are two interacting surfaces, one between IgL1 of chain A (IgL1-A, i.e. the IgL1 domain of the CD31 molecule of the first cell A) and IgL2 of chain B (IgL2-B, i.e. the IgL2 domain of the CD31 molecule of interacting cell B) and the other between IgL2 of chain A (IgL2-A, i.e. the IgL2 domain of the CD31 molecule of the first cell A) and IgL1 of chain B (IgL1-B, .e. the IgL1 domain of the CD31 molecule of interacting cell B).

The trans-homophilic interaction of CD31 domains 1 and 2 drives the clusterisation of the molecules by lateral displacement and strong cis-homophilic interaction of the transmembrane and juxtamembrane extracellular sequence.

This cis-homophilic interaction occurs at sites of cell activation and is essential for allowing the regulatory function of CD31 because this protein is not able to autophosphorylate.

CD31 phosphorylation depends on the ability of the molecules to remain clustered close to activated tyrosine kinase receptors.

Upon cell activation, the activation of cell membrane proteases drives the cleavage and shedding of most of the extracellular portion of CD31 proteins.

CD31 shedding invalidates the trans-homophilic engagement of the molecule because its trans-homophilic portion (comprised between domain 1 and 2) is lost, resulting in the dissolution of the clusters of CD31.

The amino acids included in the P8RI sequence are issued from the cis-homophilic juxta-membrane portion of CD31. P8RI co-clusters with this sequence and up-holds the regulatory signaling properties of truncated CD31 molecules in activated endothelial cells, platelets and leukocytes at site of inflammation or thrombosis.

### SUMMARY OF THE INVENTION

The inventors of the present patent application have surprisingly found some peptides endowed with the property of mimicking the trans-homophilic (domain 1 and 2) CD31-CD31 intercellular interaction.

### OBJECT OF THE INVENTION

According to a first object there are disclosed peptides having the property of mimicking the trans-homophilic CD31-CD31 domain 1 and 2 intercellular interaction.

As per preferred aspects, said peptides may have three different general structures.

As per other aspects, there are disclosed derivatives of said peptides.

According to a second object, there is disclosed the medical use of the peptides or the peptide derivatives disclosed.

As per a preferred aspect, the medical use is for the prevention of complications associated with the implantation of medical devices to treat heart and vascular pathologies.

As per another preferred aspect, the medical use is for the treatment of heart vascular pathologies.

According to a third object of the invention, it is disclosed a coating comprising the disclosed biomimetic peptides.

According to a fourth object of the invention, it is disclosed a method for the preparation of the coating comprising the disclosed biomimetic peptides.

According to a fifth object, there are disclosed devices comprising a portion coated with the peptides or the coating of the invention.

According to a sixth object, there is disclosed a method for the prevention or for the treatment or for the diagnosis of vascular pathologies comprising the use of the biomimetic peptides of the invention.

Such a use may comprise the implantation of a device coated with a peptide according to the invention.

According to another object, there is disclosed the use of the biomimetic peptides of the invention for the adhesion to the surface of a device.

As per an embodiment, there is disclosed the use of the biomimetic peptides of the invention for the adhesion to a vascular device.

According to a further object, there is disclosed the use of the biomimetic peptides of the invention for promoting the endothelialization of an arterial vessel, for preventing the neointimal growth and for the integration of a device in the target vessel.

### DETAILED DESCRIPTION OF THE INVENTION

### Definitions

For the purposes of the present invention, the term “biomimetic” is to be intended as the mimicking of a natural effect.

Within the present invention, the biomimetic peptides are endowed with the property of mimicking the natural effect of the endothelium, i.e. the layer of endothelial cells (ECs) coating the interior wall of the vessels and particular of arteries.

In particular, there is mimicked the natural effect of the endothelium under healthy conditions provided by the trans-homophilic CD31-CD31 intercellular interaction.

More in particular, said mimicking activities have the effect of not activating endothelial cells (ECs), platelets cells (circulating platelet cells) and leukocytes.

Said mimicking property can also be provided to a surface, which is covered with the peptides of the invention.

For the purposes of the present patent application, the peptides of the invention mimic the trans-homophilic CD31-CD31 domain 1 and 2 intercellular interaction.

“Homophilic interaction” shall be intended as the interaction between identical molecules, which, for the purposes of the present invention, shall be intended as the interaction of two CD31 molecules, each of them being expressed by one cell and the interacting cell, respectively.

More in particular, within each CD31 molecules, the interaction occurs through extracellular domain 1 and domain 2.

According to a first aspect, the biomimicking peptides of the invention are referred to as Group I.

In a preferred embodiment, Group I peptides span the region of human CD31 IgL1 corresponding to His71-Ser87 or a corresponding region in an ortholog mammalian CD31 molecule; alternatively, the peptides span the region of human CD31 IgL1 corresponding to Gln70-Lys89 or a corresponding region in an ortholog mammalian CD31 molecule. Amino acid sequences of ortholog mammalian CD31 molecules can be obtained from NCBI Orthologs database, available at https://www.ncbi.nlm.nih.gov/gene/5175/ortholog/?scope-40674. Corresponding regions can then be determined by a skilled person based on alignment of the CD31 ortholog amino acid sequence of interest with the human CD31 amino acid sequence.

In one embodiment, Group I peptides comprise mutated Gln70hCys and Met88hCys homocysteines that provide a disulfide bond and a cyclic structure.

Group I peptides do comprise the following sequence:

SEQ. ID no. 53
QHXXLFYKDDXXFYNISSXX

For the purposes of the present invention, within said sequence:
1Q (i.e. the residue in position 1 referred as Q in SEQ ID NO:53) may be any one of: Q, C, L, K, or R,
2H may be any one of: H, I, V, Q or R,
5L may be any one of: L, R, V, F or E,
9D may be any one of: D, E or N,
13F may be any one of: F, V, L or I,
14Y may be any one of: Y, H, R or N,
15N may be any one of: N or D,
16I may be any one of: I, V, T or A,
17S may be any one of: S or T,
18S may be any one of: S or T,
independently one from another, wherein the other amino acids X may be any other amino acid.

Consensus sequence SEQ ID NO:53 is based on the region corresponding to positions 70 to 89 of the amino acid sequence of human CD31 in a multiple sequence alignment of mammalian CD31 amino acid sequences obtained from NCBI Orthologs database on Dec. 1, 2022. Residues other than X correspond to conserved residues in mammalian ortholog CD31 amino acid sequences. Residues X correspond to highly variable positions in the multiple alignment.

Peptides of Group I comprise: SP722, SP745, SP765, SP1374, SP1375.

According to a second aspect, the biomimicking peptides of the invention mimics are referred to as Group II.

In a preferred embodiment, Group II peptides span the region of human CD31 IgL1 corresponding to Tyr107-Glu122 or a corresponding region in an ortholog mammalian CD31 molecule; alternatively, the peptides span the region of human CD31 IgL1 corresponding to 106-124 aa or a corresponding region in an ortholog mammalian CD31 molecule.

In one embodiment, mutated 106hCys and 124hCys homocysteines provide a disulfide bond and a cyclic structure.

Group II peptides do comprise the following sequence:

SEQ. ID no. 54
YKSTVIXNNKEKTTXE

For the purposes of the present invention, within said sequence:
2K (i.e. the residue in position 2 referred as K in SEQ ID NO:54) may be any one of: K or R
3S may be any one of: C or S
4T may be any one of: T, R or S
5V may be any one of: V or A
6I may be any one of: I, K, V, L, T or S
8N may be any one of: N, S or D
9N may be any one of: N, S, K or R
11E may be any one of: E, Q, V, K or M
12K may be any one of: K or R
13T may be any one of: T, A or P
14T may be any one of: T or S
16E may be any one of: E, A, Q or D
independently one from another, wherein the other amino acids X may be any other amino acid.

Consensus sequence SEQ ID NO:54 is based on the region corresponding to positions 107 to 122 of the amino acid sequence of human CD31 in a multiple sequence alignment of mammalian CD31 amino acid sequences obtained from NCBI Orthologs database on Dec. 1, 2022. Residues other than X correspond to conserved residues in mammalian ortholog CD31 amino acid sequences. Residues X correspond to highly variable positions in the multiple alignment.

Peptides of Group II comprise: SP1072, SP1376.

According to a third aspect, the biomimicking peptides of the invention are referred to as Group III.

In a preferred embodiment, Group III peptides span the region of human CD31 IgL2 corresponding to Pro133-Lys158 or a corresponding region in an ortholog mammalian CD31 molecule.

In one embodiment, Group III peptides comprise a mutated Val135hCys and Cys152hCys that provide a disulfide bond and a cyclic structure.

Group III peptides do comprise the following sequence:

SEQ. ID no. 55
PXCTLDKKEXIQGGXVXVNCSVPEEK

For the purposes of the present invention, within said sequence:
3C (i.e. the residue in position 3 referred as C in SEQ ID NO:55) may be any one of: C, V, M or I
4T may be any one of: T, I, M or E
5L may be any one of: L or V
6D may be any one of: D or N
7K may be any one of: K or R
8K may be any one of: K, T, M, R or I
11I may be any one of: I, T, M, V or E
12Q may be any one of: Q or E
14G may be any one of: G or E
16V may be any one of: V or I
18V may be any one of: V or I
19N may be any one of: N, T, R, S, G or H
22V may be any one of: V, M or L
23P may be any one of: P, Q, K, E, L or R
24E may be any one of: E, G or N
26K may be any one of: K, Q, E, R or N
independently one from another, wherein the other amino acids X may be any other amino acid.

Consensus sequence SEQ ID NO:53 is based on the region corresponding to positions 133 to 158 of the amino acid sequence of human CD31 in a multiple sequence alignment of mammalian CD31 amino acid sequences obtained from NCBI Orthologs database on Dec. 1, 2022. Residues other than X correspond to conserved residues in mammalian ortholog CD31 amino acid sequences. Residues X correspond to highly variable positions in the multiple alignment.

Peptides of Group III comprise: SP1071, SP1380.

According to a fourth aspect, the biomimicking peptides of the invention are referred to as Group IVa.

In a preferred embodiment, Group IVa peptides are heterodimers, which comprise: a) a peptide of Group II and a peptide of Group I, covalently linked.

Peptides of Group IVa comprise: SP1379.

According to a fifth aspect, the biomimicking peptides of the invention are referred to as Group IVb.

In a preferred embodiment, Group IVb peptides are heterodimers, which comprise: a) a peptide of Group III and Group I, covalently linked.

Peptides of Group IVb comprise: SP1383.

According to a first aspect, the biomimicking peptides of the invention are based on the following sequence IA:

Seq. ID. No. 4
HQMLFYKDDVLFYNISS

According to a second aspect, the biomimicking peptides of the invention are based on the following sequence IB:

Seq. ID. No. 5
YKSTVIVNNKEKTTAE

According to a third aspect, the biomimicking peptides of the invention are based on the following sequence IC:

Seq. ID. No. 6
PRVTLDKKEAIQGGIVRVNSSVPEEK

For the purposes of the present invention, the biomimicking peptides also include cyclic peptides; in particular, the biomimicking peptides comprising cysteine or homocysteine may be in the cyclic form through disulfide bond.

For the purposes of the present invention, the biomimicking peptides may comprise linkers and/or spacer and/or a tail, optionally represented by an aminoacidic sequence; in particular, said sequence may be linked to a peptidic terminus or to one amino acid residue.

In a particularly preferred embodiment, said aminoacidic sequence may be represented by the following sequence:

SEQ. ID No. 27
GGSGGSGG

For the purposes of the present invention, the biomimicking peptides also include heteropeptides comprising two of the above described peptidic sequence.

In particular, the heteropeptides may comprise:
a peptide having a sequence based on structure IA and a peptide having a sequence based on structure IB, or
a peptide having a sequence based on structure IA and a peptide having a sequence based on structure IC.

The heteropeptides may comprise peptidic sequences linked through acetyl thioether linkage between amino acids of different sequences or between linkers and/or spacers.

As per preferred aspect, the peptides of the invention are based on the following sequences:

| | Seq. |
|---|---|
| Aminoacidic sequence (IA) | ID No. |
| HQMLFYKDDVLFYNISSC | 7 |
| SHQMLFYKDDVLFYNISSS | 8 |
| QHQMLFYKDDVLFYNISSMK | 9 |
| CHQMLFYKDDVLFYNISSCK | 10 |
| YKDDVLFYNISSMKST | 11 |
| PQHQMLFYKDDVLFYNISSMK | 12 |
| FADVSTTSHVKPQHQMLFYKDDVLFYNISSMKSTESYFIPEVR | 13 |
| IY | |
| PQHQMLFYKDDVLFYNISSMKSTESYFIPEVRIYDSGTYKSTV | 14 |
| IV | |
| YKDDVLFYNISSMKSTESYFIPEVRIYDSGTYKSTVIV | 15 |
| HQMLFYKDDVLFYNISSMKSTESYFIPEVRIYDSGTYKSTVIV | 16 |
| LFYKDDVLFYNISSMKSTESYF | 17 |
| PQHQMLFYKDDVLFYNISSMK | 18 |
| QHQMLFYhCDDVLFYNISSMK | 28 |

hC = homocysteine

As per preferred aspect, the peptides of the invention are based on the following sequences:

| Aminoacidic sequence (IB) | Seq. ID No. |
|---|---|
| YKSTVIVNNKEKTTAE | 19 |
| DDVLFYNISSMK | 20 |
| QHQMLFY | 21 |
| CYKSTVIVNNKEKTTAEYC | 22 |
| hCYKSTVIVNNKEKTTAEYhC | 23 |
| GTYKSTVIVNNKEKTTAEYQ | 24 |

hC = homocysteine

As per preferred aspect, the peptides of the invention are based on the following structures:

| Aminoacidic sequence (IC) | Seq. ID No. |
|---|---|
| PRCTLDKKEAIQGGIVRVNCSVPEEK | 25 |

As per preferred aspect, the peptides of the invention are based on the following structures:

| Aminoacidic sequence | Seq. ID No. |
|---|---|
| RDQNFVILEFP | 26 |

As per a particularly preferred aspect, the heteropeptides of the invention are characterized by comprising the following peptides:

| Aminoacidic sequences | Seq. ID No. |
|---|---|
| GTYKSTVIVNNKEKTTAEYQ and | 24 and 28 |
| QHQMLFYhCDDVLFYNISSMK | (SEQ ID No. 37 |
| | and 38) |
| PRVTLDKKEAIQGGIVRVNSSVPEEK and | 6 and 28 |
| QHQMLFYhCDDVLFYNISSMK | (SEQ ID No. 39 |
| | and 40) |

According to a preferred aspect, the biomimicking peptides of the invention may comprise any of the below modifications:
a cysteine residue may be substituted by the corresponding homocysteine residue,
an L-amino acid may be substituted by the corresponding D-amino acid.

According to a preferred embodiment of the present invention, the biomimicking peptides of the invention may comprise a spacer and/or a linker and/or a tail, selected from:

| | |
|---|---|
| GGSGGSGG | SEQ. ID no. 27 |

one or more PEG4 units
one or more Ttds units
KN₃

and combinations thereof, such as for instance:

K(N₃)(Ttds)₄
(Ttds)₂-K(N₃)
(Ttds)₄-K(N₃)
K(N₃)GGSGGSGG

For the purposes of the present invention, the above disclosed structures may comprise modifications at the C-terminus and/or at the N-terminus.

In particular, said modifications may comprise:

Ac—
Br—CH₂—CO—
CH₂—CO—
CH₂—CONH₂—
Ac—KN₃
—NH₂
—CO—NH₂

According to a preferred embodiment of the invention, there are disclosed the following biomimicking peptides:

| | SEQ. | Ref |
|---|---|---|
| Aminoacidic sequence | ID No. | (SP) |
| QHQMLFYCDDVLFYNISSMK-CONH₂ | 1 | Int. A |
| (BrCH₂CO-GTYKSTVIVNNKEKTTAEYQ-(Ttds)₄-K(N₃)-NH₂) | 2 | Int. B |
| Ac-K(N₃)-(Ttds)₄-PRVTLDKKEAIQGGIVRVNSSVPEEK-Ttds-K(BrCH₂CO)- | 3 | Int. C |
| CONH₂ | | |
| Ac-c*HQMLFYKDDVLFYNISSC*-GGSGGSGG-K(N₃)-CONH₂ | 29 | 722 |
| SHQMLFYKDDVLFYNISSS-GGSGGSGG-K(N₃)-CONH₂ | 30 | 745 |
| Ac-KKKc*HQMLFYKDDVLFYNISSC*-Ttds-Ttds-K(N₃)-CONH₂ | 31 | 765 |
| QHQMLFYKDDVLFYNISSMK-Ttds-Ttds-Ttds-Ttds-K(N₃)-CONH₂ | 32 | 1375 |
| hCHQMLFYKDDVLFYNISShCK-Ttds-Ttds-Ttds-Ttds-K(N₃)-CONH₂ | 33 | 1374 |
| YKSTVIVNNKEKTTAE-PEG4-K(N₃)-CONH₂ | 34 | 1072 |
| Ac-K(N₃)-PEG4-PRVTLDKKEAIQGGIVRVNSSVPEEK-CONH₂ | 35 | 1071 |
| Ac-K(N₃)-(Ttds)₄-PRhCTLDKKEAIQGGIVRVNhCSVPEEK-CONH₂ | 36 | 1380 |
| [^{#}CH₂CONH₂GTYKSTVIVNNKEKTTAEYQ-(Ttds)₄-K(N₃)-NH₂]- | 37 and 38 | 1379 |
| [QHQMLFYhC^{#}DDVLFYNISSMK-CONH₂] | | |
| [Ac-K(N₃)-(Ttds)₄-PRVTLDKKEAIQGGIVRVNSSVPEEK-Ttds-K^{#}(CH₂CO)- | 39 and 40 | 1383 |
| CONH₂]-[QHQMLFYhC^{#}DDVLFYNISSMK-CONH₂] | | |
| Ac-K(N₃)-GGSGGSGG-YKDDVLFYNISSMKST-NH₂ | 41 | 547 |
| Ac-YKDDVLFYNISSMKST-GGSGGSGG-K(N₃)-NH₂ | 42 | 548 |
| Ac-PQHQMLFYKDDVLFYNISSMK(GGSGGSGG-K(N₃))STESYFI-CONH₂ | 43 | 724 |
| Ac-K(N₃)-GGSGGSGG- | 44 | 732 |
| FADVSTTSHVKPQHQMLFYKDDVLFYNISSMKSTESYFIPEVRIYDSGTYK-CONH₂ | | |
| Ac-K(N₃)-GGSGGSGG- | 45 | 723 |
| PQHQMLFYKDDVLFYNISSMKSTESYFIPEVRIYDSGTYKSTV-CONH₂ | | |
| Ac-K(N₃)-GGSGGSGG- | 46 | 746 |
| YKDDVLFYNISSMKSTESYFIPEVRIYDSGTYKSTVIV-CONH₂ | | |
| Ac-K(N₃)-GGSGGSGG- | 47 | 747 |
| HQMLFYKDDVLFYNISSMKSTESYFIPEVRIYDSGTYKSTVIV-CONH₂ | | |
| Ac-LFYKDDVLFYNISSMKSTESYF-GGSGGSGG-K(N₃)-CONH₂ | 48 | 762 |
| Ac-LFYKDDVLFYNISSMKSTESYF-Ttds-Ttds-K(N₃)-CONH₂ | 49 | 763 |
| Ac-PQHQMLFYKDDVLFYNISSMK(GGSGGSGG-K(N₃))STESYFIKK-CONH₂ | 50 | 766 |
| RDQNFVILEFP-PEG4-K(N₃)-NH₂ | 51 | 1070 |

wherein
hC: homoCysteine; c: D-Cysteine; **position for disulfide bridge formation of cyclic
peptides
^{#}acetyl thioether linkage

For the purposes of the present invention, the biomimicking peptides of the invention comprise peptides having at least 95% identity with any one the above disclosed structures, preferably at least 97% and even more preferably at least 99% identity. The percent identities referred to in the context of the disclosure of the present invention are determined after optimal global alignment of the sequences to be compared, which optimal global alignment may therefore comprise one or more insertions, deletions, truncations and/or substitutions. The alignment is global, meaning that it includes the sequences to be compared taken in their entirety over their entire length. The alignment is “optimal”, meaning that the number of insertions, deletions, truncations and/or substitutions is made as low as possible. The optimal global alignment may be performed and the percent identity calculated using any sequence analysis method well-known to the person skilled in the art. In addition to manual comparison, it is possible to determine global alignment using the algorithm of Needleman and Wunsch (1970). For nucleotide sequences, the sequence comparison may be performed using any software well-known to a person skilled in the art, such as the Needle software. The parameters used may notably be the following: “Gap open” equal to 10.0, “Gap extend” equal to 0.5, and the EDNAFULL matrix (NCBI EMBOSS Version NUC4.4). For amino acid sequences, the sequence comparison may be performed using any software well-known to a person skilled in the art, such as the Needle software. The parameters used may notably be the following: “Gap open” equal to 10.0, “Gap extend” equal to 0.5, and the BLOSUM62 matrix.

According to a second object, there is disclosed the medical use of the peptides disclosed.

As per a preferred aspect, the medical use is for the prevention of vascular pathologies.

Such medical use may comprise the implantation of a device coated with a peptide according to the invention.

In particular, according to the present invention, said pathologies are selected from the group comprising: heart valve pathology, atherosclerosis, thrombosis, ischemia, hemorrhage, restenosis, aneurysm.

In addition to the above, there is disclosed the medical use for the prevention of a pathological condition represented by the in-stent stenosis.

As per another preferred aspect, the medical use is for the treatment of vascular pathologies.

The above medical uses are disclosed for humans as well as for animals in the veterinary field.

According to a third object of the invention, it is disclosed a coating comprising the disclosed biomimetic peptides.

As per an embodiment of the present invention, the coating is a mono-layered coating, while as per an alternative embodiment of the invention it is a multi-layer coating.

According to a fourth object of the invention, it is disclosed a method for the preparation of the coating comprising the disclosed biomimetic peptides.

For the purposes of the present invention, the coating of a device or a portion thereof can be obtained with dip-coating, by immersion of the portions to be coated within a coating bath.

A well-known method for functionalizing a surface is click-chemistry, as for example disclosed in US 2018/0296732, WO 2020/109836 or WO 2021/239905.

Copper-free click chemistry is based on the reaction of a diarylcyclooctyne moiety (DBCO, also known as ADIBO for azadibenzocyclooctyne or DIBAC for dibenzoazacyclooctyne) with an azide-labeled reaction partner, known as strain-promoted alkyne azide cycloaddition (SPAAC). This click chemistry is very fast at room temperature and does not require a Cu (I) catalyst. Diarylcyclooctynes are thermostable with very narrow and specific reactivity toward azides, resulting in almost quantitative yields of stable triazole. The reaction can be carried out in aqueous buffered media.

As per a preferred embodiment, the method is a three-step dip coating method comprising:
A first step for a polydopamine coating,
A second step for grafting a suitable linker, and
A third step for the coating with the invention peptide.

The first step represents the realization of a coating of a polydopamine layer onto the surface of the device, or of a portion thereof, in order to obtain a polydopamine coated surface from dopamine.

Dopamine is known to self-polymerize into a very adherent film, on several kinds of substrates. Polydopamine (hereafter PDA) is a self-assembling polymer formed by the oxidation of the dopamine. Indeed, PDA contains dopamine, indole, and pyrrole units. Due to its variety of reactive groups, PDA is known to provide several possibilities for substrates functionalization, especially for bioactive molecule immobilization. PDA coating can be performed by dipping the device or a portion thereof in a solution comprising a salt of dopamine, notably dopamine hydrochloride or dopamine ammonia. The solution can be an aqueous solution, an alcoholic solution or an aqueous-alcoholic. The solvent of the solution is preferably an alcohol, in particular absolute ethanol, when the device is sensible to water (corrosion for instance). The device can be pre-treated before immersion in the dopamine solution, for example by etching with a strong acid such as hydrofluoric acid.

At the end, a rinsing step and/or sonication step can be done in order to remove the polydopamine uncoated or PDA aggregates. The rinsing step can be done with demineralized water or alcohol.

The second step corresponds to the immobilization of a linker onto the polydopamine coating in order to subsequently graft a peptide of interest. Any linker allowing the immobilization of the biomimetic peptide can be used. For that purpose, the polydopamine coating previously obtained in the first step is put in contact, by any suitable means, with a linker solution in order to form a linker film on the polydopamine coating.

The linker is any suitable bifunctional reagent. The linker is preferably bio-orthogonal.

The linker is preferably suitable for click chemistry as disclosed above. Accordingly, the linker comprises a free triple bond, preferably a cyclooctyne moiety, more preferably a diarylcyclooctyne moiety such as DBCO. The triple bond can react with the azide group of the peptide. The linker is advantageously further functionalized with amine and/or thiol functional groups which are reactive towards polydopamine coatings.

To enhances solubility in water as well as in commonly used organic solvents of moderate polarity, the linker preferably comprises a spacer. This spacer is preferably a polymer or oligomer. The polymer or oligomer that may be used include polyethylene glycol (PEG), polylactate, polylactic acids, sugars, lipids, polyglutamic acid (PGA), polyglycolic acid, poly(lactic-co-glycolic acid) (PLGA), polyvinyl acetate (PVA), and the combinations thereof. PEG is particularly preferred. Specifically, the PEG4 hydrophilic spacer is known to reduce or eliminate aggregation or precipitation problems.

Examples of cyclooctyne PEG are DBCO-PEG derivatives, like DBCO-PEG4-amine such as DBCO-sulfo-PEG4-NH₂.

A rinsing step is preferably done in order to remove the linker not immobilized on the surface of PDA coating. The rinsing step can be done with demineralized water and/or alcohol.

The third step comprises the grafting of the biomimetic peptide onto the modified polydopamine coated device with a suitable linker. For that purpose, the peptide preferably comprises a functional group, such as an azide group, able to react with the functional group of the linker, such as a free triple bond.

The device, or a portion thereof, obtained after the second step is put in contact, by any suitable means, with the biomimetic peptide comprising a specific functional group in order to graft it on the device. The grafting can be performed by dipping the device or a portion thereof in an aqueous solution comprising the biomimetic peptide. The reaction can be performed at room temperature.

According to a fifth object of the invention, there are disclosed devices comprising a portion coated with the biomimetic peptides or the coating of the invention.

Coating may comprise the whole surface of a device or of a part of a device.

For the purposes of the present invention, devices, which can be fully or partially covered with biomimetic peptides of the invention comprise: coronary stent, flow diverting stent, aortic tubes, cardiac valves, balloon expandable stents, self-expandable scaffolds, the polymer tube of graft-stents, flow diverting meshes, aortic tubes, cardiac valves, stent retriever, transcatheter mitral valve device, catheter, leaflet or any portions thereof, in particular balloon expandable stents, self-expandable scaffolds, the polymer tube of graft-stents, flow diverting meshes, aortic tubes, cardiac valves, stent retriever, transcatheter mitral valve device, catheter, leaflet or any portions thereof, and in general any medical device that can be in contact with the blood for a limited or extended period of time.

The fully or partially coated devices may be in any suitable material, such as, for instance:
Metal or alloys: stainless steel, cobalt-chromium alloy, platinum-chromium alloy, nickel-titanium alloy (also known as nitinol), cobalt-chromium-nickel alloy, magnesium or magnesium alloys such as JDBM, Mg—Nd—Zn—Zr alloy, Mg—Nd—Zn—Ca alloy, Mg—Zn—Y—Nd alloy, Mg—Al, Mg-AL-Zr, WE43, AZ31;
Synthetic polymeric materials: PEBAX, PVP, PE, PP, Dacron®, Teflon®;
Clinical-grade biological tissues, such as pericardial sheets for heart valves bioprostheses.

According to a sixth object, there is disclosed a method for the prevention or for the treatment of heart and vascular pathologies comprising the use of the biomimetic peptides of the invention.

According to a particular aspect, said method for the prevention or for the treatment of heart and vascular pathologies comprises the use of a device fully or partially coated with the coating of the invention. In particular, the devices can be balloon-mounted stents, heart valve bioprotheses, flow diverters and the said devices can be used in interventional cardiovascular procedures, such as for coronary and peripheral artery revascularization, interventional neurology and heart valve implantation by instance.

In particular, targeted pathologies are represented by: heart valve pathology, stenotic vascular disease (including arteriosclerosis and in particular atherosclerosis), atherothrombosis, ischemic heart and peripheral disease, vessel remodeling exposing to the risk of hemorrhage, restenosis, aneurysm.

In addition to the above, there is disclosed a method for the prevention of a condition represented by the in-stent stenosis.

It is well known that one of the early mechanisms driving a pathologic tissue remodelling linked to the presence of a foreign body in contact with the blood is the adhesion of platelets to surfaces of the said foreign body, which triggers the recruitment and activation of leukocytes then triggering the onset of a cascade of reactions leading potentially to the formation of a blood clot and/or to the encapsulation of the foreign body by vascular cells rapidly proliferating in response to soluble factors released by the activated platelets and leukocytes, eventually resulting in the reduction of the vessel lumen, e.g. the occurrence of (re) stenosis (see for example: Forrester et al., J Am Coll Cardiol., 17:758-769 (1991)).

It is for such reasons that in the context of heart and vascular pathologies, individuals into whom medical devices such as stents and valve bioprotheses are implanted are generally given antiplatelet therapy (aspirin and/or anti-P2Y12 therapy, such as clopidogrel, ticlopidine, ticagrelor, or prasugrel) at least for one to several months after stent implantation. The use of one and more often of two anti-platelet treatments is intended to eliminate the risk of platelet activation at the contact with the stent that is exposed to the blood flow until fully re-endothelialized, and thus to eliminate the risk of stent thrombosis and limit the rate of restenosis. Most of the time, dual antiplatelet therapy (DAPT) comprising aspirin and an anti-P2Y12 therapy is administered during 6 to 12 months. Such a treatment eventually exposes the patient at the risk of hemorrhage and this observation has prompted to evaluate the safety of a reduced duration of the antiplatelet treatment. Clinical trials with drug eluting stents in which the DAPT is reduced to 30 days are currently ongoing. Once the period requiring a DAPT is over, the anti-platelet treatment generally consists in a continued therapy with aspirin alone (without anti-P2Y12 therapy).

When using drug-eluting stents, the recommended dose of aspirin (acetyl-salicylic acid or a salt thereof) in humans is between 50 and 100 mg/day, such as 75 mg/day. Regarding anti-P2Y12 agents, the recommended dose in humans varies depending on the specificanti-P2Y12 agent used. The recommended dose for clopidogrel is between 50 and 100 mg/day, in particular 75 mg/day (generally 75 mg once a day). The recommended dose for ticlopidine is between 200 and 300 mg/day, in particular 250 mg/day (generally 250 mg once a day). The recommended dose for ticagrelor is between 160 and 200 mg/day, in particular 180 mg/day (generally 90 mg twice a day). And the recommended dose for prasugrel is between 5 and 20 mg/day, in particular 10 mg/day (generally 10 mg once a day).

However, antiplatelet therapy may be associated to significant risk of adverse effects and difficulties in any individual, mainly hemorrhage, and even more in individuals suffering from bleeding disorders, such as hemophilia A (factor VIII deficiency), hemophilia B (factor IX deficiency), von Willebrand disease, and rare factor deficiencies including I, II, V, VII, X, XI, XII and XIII. In particular, anti-P2Y12 agents are very powerful molecules, that tend to provoke bruises all over the body, for no specific reason. Moreover, no dentist, gastroenterologist, or surgeon will want to touch a patient while under anti-P2Y12 therapy, to stay away from any risk of unmanageable bleeding upon intervention. In some cases, this can be a severe problem as it prevents intervention on some diseases other than cardiovascular, that would normally require intervention. These molecules also have notable side effects, including diarrhea, itching, nausea, skin rash, and stomach pain.

Based on the physiologic, non-activated, endothelial tissue-mimicking properties of the coating made of CD31-derived peptides onto the medical device according to the invention, the initial adhesion/activation of blood platelets entering in contact with the device may be prevented or strongly reduced, making the anti-platelets treatment that usually follows the implantation of said device either unnecessary or at least reduced to lower doses and/or administered during a shorter timeframe than usual.

Therefore, in the context of any method for the prevention or for the treatment of heart and vascular pathologies (including heart valve pathology, atherosclerosis, atherothrombosis, ischemia, hemorrhage, restenosis, aneurysm) or of any method for the prevention of a condition represented by the in-stent stenosis, after the implantation with the medical device according to the invention, the recipient individual preferably:
a) does not take anti-P2Y12 therapy (the individual may even not take any antiplatelet therapy);
b) takes a significantly lower (e.g. at least twice, at least 3 times, at least 4 times . . . ) dose of anti-P2Y12 therapy (and optionally also a significantly lower, such as least twice, at least 3 times, at least 4 times . . . lower dose of aspirin) than recommended for drug-eluting stents within a traditional dual anti-platelet therapy (DAPT);
c) takes anti-P2Y12 therapy (and optionally aspirin) during a significantly shorter (e.g. at least twice, at least 3 times, at least 4 times . . . shorter) timeframe than recommended for drug-eluting stents within a traditional dual anti-platelet therapy (DAPT); or
d) any combination of b) and c).

All of items a) to d) above only relate to the treatment post-implantation of the medical device (stent in particular), and not to drugs that may be administered during implantation of the medical device, which are chosen by the physician in accordance with clinical recommendations.

In items b), c) and d) above, “drug-eluting stents” or “DES” refers to stents that slowly release a drug inhibiting the proliferation of smooth muscle cells (SMC), such as sirolimus. Examples of commercially available drug-eluting stents include HT Supreme®, Xience VR, Promus®, Cypher®, Taxus®, and Endeavor®.

In item b) above, after implantation the individual into which the medical device according to the invention is implanted takes a significantly lower (e.g. at least twice, at least 3 times, at least 4 times . . . ) dose of anti-P2Y12 therapy than recommended for drug-eluting stents within a traditional dual anti-platelet therapy (DAPT). In particular, after implantation, the individual may take:
less than 50 mg/day, preferably less than 40 mg/day, less than 35 mg/day, less than 30 mg/day, less than 25 mg/day or even less than 20 mg/day of clopidogrel,
less than 200 mg/day, preferably less than 175 mg/day, less than 150 mg/day, less than 125 mg/day, or even less than 100 mg/day of ticlopidine,
less than 160 mg/day, preferably less than 150 mg/day, less than 140 mg/day, less than 130 mg/day, less than 120 mg/day, less than 110 mg/day, less than 100 mg/day, less than 90 mg/day or even less than 80 mg/day of ticagrelor (generally taking half of the daily dose twice a day), or
less than 5 mg/day, preferably less than 4 mg/day, less than 3 mg/day, less than 2.5 mg/day, or even less than 2 mg/day of prasugrel.

The individual may further take after implantation a significantly lower (e.g. at least twice, at least 3 times, at least 4 times . . . ) dose of aspirin. For instance, the individual may take less than 50 mg/day, preferably less than 40 mg/day, less than 35 mg/day, less than 30 mg/day, less than 25 mg/day or even less than 20 mg/day of aspirin.

In item c) above, after implantation the individual into which the medical device according to the invention is implanted takes anti-P2Y12 therapy during a significantly shorter (e.g. at least twice, at least 3 times, at least 4 times . . . shorter) timeframe than recommended for drug-eluting stents. The individual may further take aspirin after implantation during a significantly shorter (e.g. at least twice, at least 3 times, at least 4 times, . . . shorter) timeframe than recommended for drug-eluting stents.

For instance, the individual may take DAPT during less than 3 months, preferably less than 2 months, less than 1 month, or even less than 4 weeks, less than 3 weeks, less than 2 weeks or less than 1 week. A shorter antiplatelet treatment may also mean that the individual completely stops anti-P2Y12 therapy after a certain duration of treatment.

Item d) above is any combination of items b) and c) above.

This revised protocol without or with less antiplatelet therapy is useful in any individual, as it prevents or strongly inhibit adverse effects (bleeding events) associated to antiplatelet therapy. It is however particularly useful for individuals with bleeding disorders, such as hemophilia A (factor VIII deficiency), hemophilia B (factor IX deficiency), von Willebrand disease, and rare factor deficiencies including I, II, V, VII, X, XI, XII and XIII.

The above disclosed method for the prevention or for the treatment are disclosed for humans as well as for animals in the veterinary field.

According to another object, there is disclosed the use of the biomimetic peptides of the invention for the adhesion to the surface of a device.

As per an embodiment, there is disclosed the use of the biomimetic peptides of the invention for the adhesion to vascular device.

According to a further object, there is disclosed the use of the biomimetic peptides of the invention for promoting the endothelization of a vessel, for preventing the neointimal growth and for integration of a device in the target vessel.

In particular, a target vessel is represented by a coronary arterial vessel, a peripheral arterial vessel or a cerebral vessel.

According to an even further object, there is disclosed the use of the biomimetic peptides of the invention for improving the biocompatibility of a device.

According to an even further object, there is disclosed the use of the biomimetic peptides of the invention for endowing the stented segment with recovered endothelial anti-inflammatory and anti-thrombotic properties.

According to an even further object, there is disclosed the use of the biomimetic peptides of the invention for endowing the stented segment with improved adaptive remodelling allowing recovery of its functional properties.

The present invention will be further disclosed in the following non-limitative examples.

### EXAMPLES

### BRIEF DESCRIPTION OF THE FIGURES

FIGS. 1A and 1B show the structure of the octa-peptide P8RI.

FIG. 2 shows the strategy used to analyze the biocompatibility of nitinol disks interacting with human endothelial cells.

FIG. 3 shows representative images and quantification of the F-actin staining of HAEC growing onto bare and coated disks.

FIG. 4 shows representative images and quantification of the CD31 staining of HAEC growing onto bare and coated disks.

FIG. 5 shows quantitative analysis of F-actin and CD31 expression on HAEC growing onto coated vs bare control nitinol disks.

FIG. 6 shows the ratio between CD31 and F-actin expression.

FIGS. 7 to 17 show the structure of the preferred peptides of the invention.

FIG. 18 shows the structure of the intermediate A.

FIG. 19 shows the structure of the intermediate B.

FIG. 20 shows the structure of the intermediate C.

FIG. 21 shows the structures of linkers according to the present invention.

FIG. 22 shows the results of the functional score of peptides of the invention belonging to different groups.

FIG. 23 shows a graph representing the results of the functional score of peptides of the invention belonging to different groups.

FIG. 24 shows a graph representing the biomimetic performances of the peptides of the invention belonging to different groups.

FIG. 25 shows the ratio between CD31 and F-actin expression.

FIG. 26 shows the infra-red spectrum of the eG™ NTMA film.

FIG. 27 shows the shapes of a water droplet on the bare surface and on the eG™ NTMA surface.

FIG. 28 shows SEM cross section, 7 days after CFD stents implantation, with eG NTMA vs SP1072 peptide coating.

FIG. 29 shows histopathology analysis of SEM cross section, 7 days after CFD stents implantation, with eG NTMA vs SP1072 peptide coating.

FIG. 30 shows histopathology analysis of SEM cross section, 60 days after CFD stents implantation, with SP1072 peptide coating.

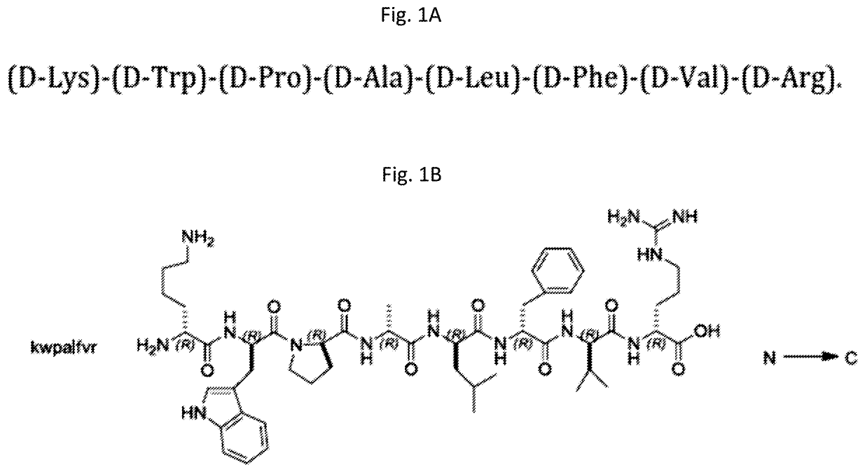

### ABBREVIATIONS

Certain abbreviations are used in the examples and elsewhere herein:
“AA” refers to amino acid;
“Alloc” refers to allyloxycarbonyl
“Boc” refers to tert-butyloxycarbonyl;
“tBu” refers to tertiary butyl;
“DCM” refers to dichloromethane;
“DIC” refers to N,N′-diisopropylcarbodiimide;
“DIPEA” refers to N, N-Diisopropiletylamine
“DMF” refers to dimethylformamide;
“Fmoc” refers to fluorenylmethyloxycarbonyl;
“HOAt” refers to 1-hydroxy-7-azabenzotriazole;
“HPLC” refers to High Performance Liquid Chromatography;
“LCMS” refers to Liquid Chromatography/Mass Spectrometry;
“UPLC” refers to High Performance Chromatography;
“RP-HPLC” refers to reversed-phase high performance liquid chromatography;
“MS” refers to mass spectrometry;
“OtBu” refers to O-tert-butyl;
“Oxyma” refers to ethyl cyanohydroxyiminoacetate;
“Pbf” refers to 2,2,4,6,7-pentamethyldihydrobenzofuran-5-sulfonyl;
“TIPS” refers to triisopropylsilane;
“TFA” refers to trifluoroacetic acid;
“Trt” refers to trityl

### Materials and Methods

The peptides were synthesized by standard Solid-phase Peptide Synthesis (SPPS) using Fmoc/t-Bu chemistry. DMF was used as the solvent. The following starting materials and methods were employed in the synthetic procedures described in the examples.

Fmoc-protected natural amino acids were purchased from Novabiochem, Iris Biotech, Bachem or Chem-Impex International. The following standard amino acids were used in the syntheses: Fmoc-L-Ala-OH, Fmoc-L-Arg (Pbf)-OH, Fmoc-L-Asn (Trt)-OH, Fmoc-L-Asp (OMpe)-OH, Fmoc-L-Gln (Trt)-OH, Fmoc-L-Glu (OtBu)—OH, Fmoc-L-Gly-OH, Fmoc-L-Ile-OH, Fmoc-L-Leu-OH, Fmoc-L-Lys (Boc)-OH, Fmoc-L-Pro-OH, Fmoc-L-Ser (tBu)—OH, Fmoc-L-Thr (tBu)—OH, Fmoc-L-Tyr (tBu)—OH, Fmoc-L-Val-OH.

In addition, the following amino acids were purchased from the same suppliers as above: Fmoc-K—(N₃) OH; Fmoc-K (alloc) OH; Fmoc-c-OH, Fmoc-hC-OH, Fmoc-Ttds-COOH, Fmoc-NH-PEG4-COOH.

### Analytical Characterization.

Crude and purified peptides were analyzed by Ultra-high performance liquid chromatography with UV and mass spectrometry detection (UPLC-UV-MS). Analytical UPLC was performed according to one of the following methods:

### Method A:

Detection at 214 nm
Column: Acquity Waters BEH130 C4, 1.7 μm (2.1×100 mm) at 45° C.
Solvent: H₂O+0.1% TFA: ACN+0.1% TFA (flow 0.4 ml/min)
Gradient: 85:15 (0 min) to 85:15 (1 min) to 65:35 (5 min) to 10:90 (5.2 min) to 10:90 (5.5 min) to 85:15 (5.7 min) to 85:15 (6 min)
Mass analyzer: Waters SQ Detector with electrospray ionization in positive ion detection mode

### Method B:

Detection at 214 nm
Column: Acquity Waters BEH130 C4, 1.7 μm (2.1×100 mm) at 45° C.
Solvent: H₂O+0.1% TFA: ACN+0.1% TFA (flow 0.4 ml/min)
Gradient: 80:20 (0 min) to 80:20 (1 min) to 60:40 (5 min) to 10:90 (5.2 min) to 10:90 (5.5 min) to 80:20 (5.7 min) to 80:20 (6 min)

### Method C:

Detection at 214 nm
Column: Acquity Waters BEH130 C4, 1.7 μm (2.1×100 mm) at 45° C.
Solvent: H₂O+0.1% TFA: ACN+0.1% TFA (flow 0.4 ml/min)
Gradient: 75:25 (0 min) to 75:25 (1 min) to 55:45 (4 min) to 10:90 (4.2 min) to 10:90 (4.5 min) to 80:20 (4.7 min) to 75:25 (5 min)
Mass analyzer: Waters SQ Detector with electrospray ionization in positive ion detection mode

Mass analysis was performed on a Waters SQ Detector with electrospray ionization in positive ion detection mode and the scan range of the mass-to-charge ratio was 400-1800.

TABLE 1
Analytical method, retention time, calculated and found masses, of the example compounds

| | | Exact Mass | Observed | Observed | Observed | Observed | Retention | |
|---|---|---|---|---|---|---|---|---|
| SP | SEQ ID no. | (monoisotopic) | [M + 2H]2+ | [M + 3H]3+ | [M + 4H]4+ | [M + 5H]5+ | Time (min) | Method |
| 722 | 29 | 3034.32 | 1519.2 | 1013.5 | ND | ND | 3.27 | C |
| 745 | 30 | 2962.37 | 1483.8 | 989.4 | ND | ND | 2.85 | C |
| 765 | 31 | 3506.78 | 1756.2 | 1171.0 | 878.6 | ND | 2.90 | C |
| 1375 | 32 | 3868.04 | ND | 1291.8 | 969.2 | 775.5 | 4.20 | B |
| 1374 | 33 | 3843.58 | ND | 1282.8 | 962.4 | 770.2 | 4.05 | B |
| 1072 | 34 | 2225.21 | 1114.0 | 743.0 | ND | ND | 2.97 | A |
| 1376 | 23 | 3580.90 | ND | 1195.4 | 897.1 | ND | 3.57 | A |
| 1071 | 35 | 3249.83 | ND | 1085.0 | 814.0 | 651.6 | 3.65 | A |
| 1380 | 36 | 4298.36 | ND | 1435.4 | 1076.8 | 861.8 | 3.23 | B |
| 1379 | 37-38 | 6169.14 | ND | ND | 1545.5 | ND | 4.35 | B |
| 1383 | 39-40 | 7216.85 | ND | ND | ND | 1445.9 | 4.59 | B |

ND: Not Detected

### General Procedure for Peptide Synthesis on Solid Support

The synthesis of all the peptides was performed by standard Fmoc stepwise solid phase synthesis (SPPS) on a Liberty Blue microwave synthesizer (CEM corp.). The assembly was performed using a Protide Rink-amide (4-(2′,4′-Dimethoxyphenyl-Fmoc-aminomethyl)-phenoxyacetamido-norleucylaminomethyl resin, CEM, 200 μmol, 100-200 Mesh; loading 0.2 mmol/g) on a 0.2 mmol scale, with DIC/Oxyma activation. DMF was used as the solvent.

All the amino acids were dissolved at a 0.4 M concentration in DMF. The acylation reactions were performed for 3 min at 90° C. under microwave irradiation with 5 folds excess of activated amino acids over the resin free amino groups. The amino acids were activated with equimolar amounts of 0.5 M solution of DIC in DMF and Oxyma solution 1 M in DMF.

The following conditions were employed:
Standard deprotection: 20% piperidine in DMF for 2×120 s, 90° C.
Washes: 4×DMF.
Standard Single coupling: 5 eq. AA 0.4 M/5 eq. DIC 1M/5 eq. Oxyma 1M, 120 s, 90° C.
Standard Double coupling: standard single coupling repeated twice.
Washes: 4×DMF.

At the end of the assembly the resin was washed with DMF, MeOH, DCM, Et₂O.

Cleavage of the peptides from the resin was performed using the following cleavage cocktail:

Mix 1) 87.5% TFA, 5% phenol, 5% water, 2.5% TIPS for 1.5 to 2.5 hours at RT (30 ml).

Mix 2) 87.5% TFA, 5% phenol, 5% water, 2.5% Thioanisole for 1.5 to 2.5 hours at RT (20 ml).

The resin employed in the synthesis was such that the C-terminal was cleaved from the resin as a primary amide.

The cleavage mixture was collected by filtration, the crude peptides were precipitated in methyl tert-butyl ether, centrifuged, the supernatant was removed, fresh diethyl ether was added to the peptides and re-centrifuged, twice; the crude peptides were then lyophilized.

Peptides were analyzed by analytical UPLC and verified by ESI+ mass spectrometry. Crude peptides were purified by a conventional preparative RP-HPLC purification on a preparative Waters 2489 HPLC system, (UV detection at wavelength 214 nm); the following solvents as eluents: acetonitrile+0.1% TFA (mobile phase A) and water+0.1% TFA (mobile phase B). Product containing fractions were collected and lyophilized to obtain the purified product as a TFA salt. Unless otherwise described the compounds were tested as TFA salt.

Procedure for the synthesis of Seq ID 29:
Ac-c*HQMLFYKDDVLFYNISSC*-GGSGGSGG-K(N₃)-CONH₂

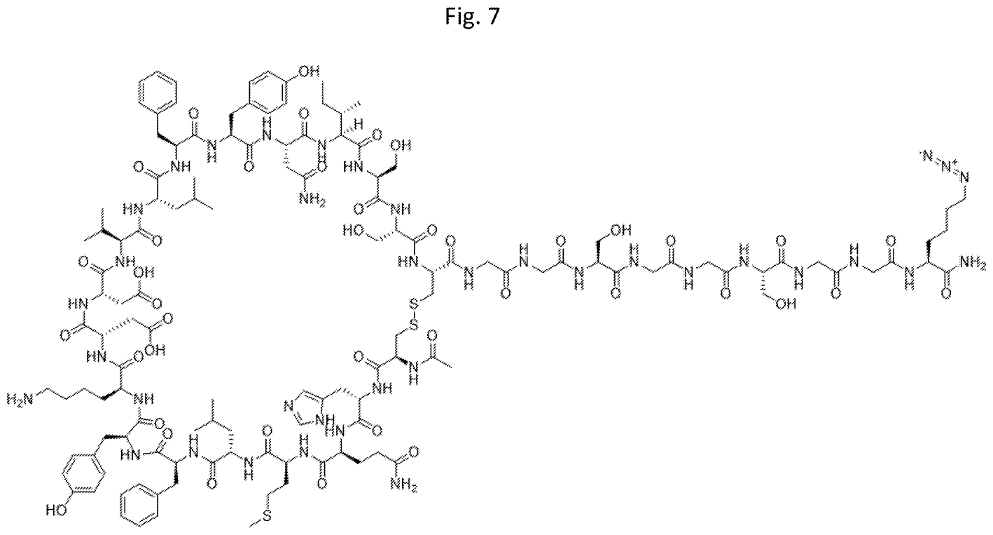

The structure of the peptide is shown in FIG. 7.
Seq ID 29 (PepSP722) spans region His71-Ser87 of CD31 IgL1-A with an acetylated N-terminal D-Cysteine and a Cysteine at the C-terminal position of Ser87, with the two thiol groups engaged in a disulfide bond.
After the synthesis and the cleavage performed according to the general procedure (Mix 1), the mixture was analyzed by analytical UPLC and verified by ESI+ mass spectrometry and it was lyophilized to afford the crude title compound (Y=64%). Crude material was dissolved in a mixture of 8/2 acetonitrile/water at 1 mg/mL concentration: aqueous NH₃ added to reach pH 9 and the mixture was stirred for 72 hours at room temperature. TFA added to the mixture and freeze dried. The crude peptide was purified by reverse-phase HPLC using preparative Reprosil Gold C4 (250×40 mm, 120 Å, 5 μm) column. The following gradient of eluent B was used: 20% B to 20% B over 5 min, to 40% B over 25 min, flow rate 60 mL/min, wavelength 214 nm. Seq ID 1 was isolated as an amorph white freeze-dried solid (Y=5%), TFA salt. The purified peptide was solubilized at 1 mg/mL concentration in a mixture of acetonitrile/water 8/2: HCl 50 mM (10 eq) was added, and mixture stirred for 1 hour at room temperature. After lyophilization, the peptide was dissolved in acetonitrile/water 8/2 and freeze dried again. The peptide was analyzed by LC/MS (Method C). [M+3H]³⁺ mass signal found under the peak with retention time 3.27 min revealed the peptide mass 1013.5 which is in line with the expected value of molecular weight 3036.37.

Procedure for the synthesis of Seq ID 30:
SHQMLFYKDDVLFYNISSS-GGSGGSGG-K(N₃)-CONH₂

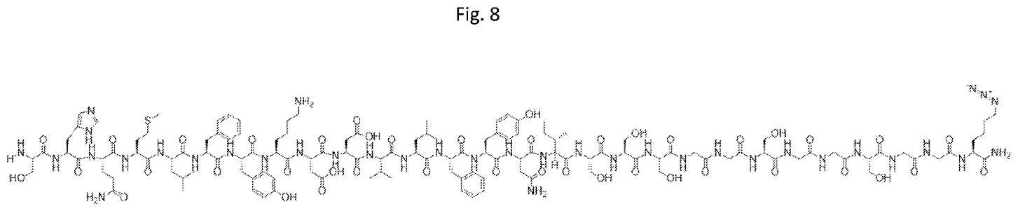

The structure of the peptide is shown in FIG. 8.
Seq ID 30 (PepSP745) spans the same region His71-Ser87 of CD31 IgL1-A, common with Seq ID 29 but it is a linear sequence with replacement of the two cysteine residues in seq ID1 in Serines.
After the synthesis and the cleavage performed according to the general procedure (Mix 1), the mixture was analyzed by analytical UPLC and verified by ESI+ mass spectrometry and it was lyophilized to afford the crude title compound (Y=66%). The crude peptide was purified by reverse-phase HPLC using preparative Reprosil Gold C4 (250×40 mm, 120 Å, 5 μm) column. The following gradient of eluent B was used: 20% B to 20% B over 5 min, to 40% B over 20 min, flow rate 60 mL/min, wavelength 214 nm. Seq ID 2 was isolated as an amorph freeze-dried solid (Y=4%), TFA salt. The peptide was solubilized at 1 mg/mL concentration in a mixture of acetonitrile/water 8/2: HCl 50 mM (10 eq) was added, and mixture stirred for 1 hour at room temperature. After lyophilization, the peptide was dissolved in acetonitrile/water 8/2 and freeze dried again. The peptide was analyzed by LC/MS (Method C). [M+3H]³⁺ mass signal found under the peak with retention time 2.85 min revealed the peptide mass 989.4 which is in line with the expected value of molecular weight 2964.22.

Procedure for the synthesis of Seq ID 31:
Ac-KKKc*HQMLFYKDDVLFYNISSC*-Ttds-Ttds-K(N₃)-CONH₂

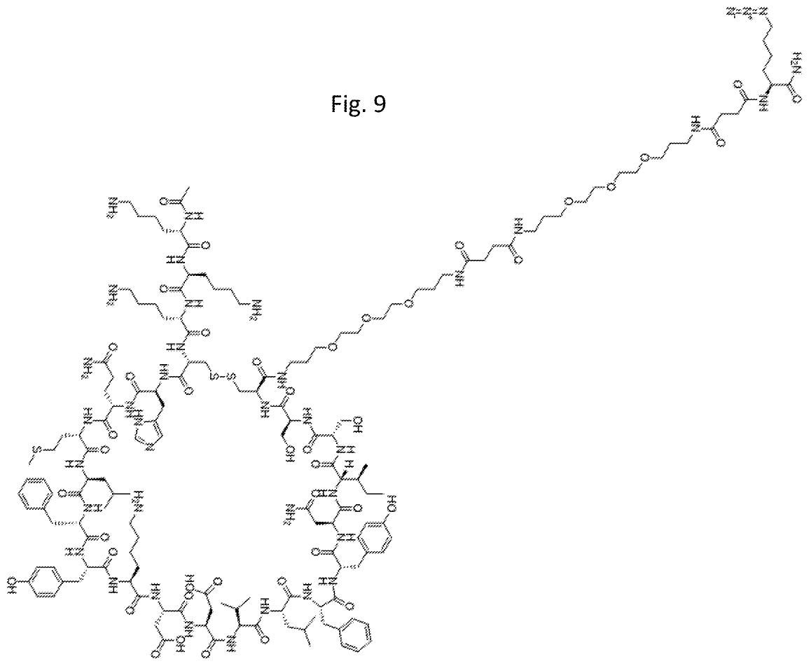

The structure of the peptide is shown in FIG. 9.
Seq ID 31 (PepSP765) spans the same region His71-Ser87 of CD31 IgL1-A and similar disulfide connectivity of seq ID 29 but with a solubilizing tail at the N-terminus of 3 lysine residues.
The synthesis was performed according to the general procedure. At the end of the assembly the resin was treated with Ac₂O (10 eq) in DMF for 30 mins. The cleavage was performed according to the general procedure using Mix 1, the mixture was analyzed by analytical UPLC and verified by ESI+ mass spectrometry and freeze-dried (Y=61%). Crude material was dissolved in a mixture of 8/2 acetonitrile/water at 1 mg/mL concentration: aqueous NH₃ added to reach pH 9 and the mixture was stirred for 72 hours at room temperature. TFA added to the mixture and freeze dried. The crude peptide was purified by reverse-phase HPLC using preparative Reprosil Gold C4 (250×40 mm, 120 Å, 5 μm) column. The following gradient of eluent B was used: 25% B to 25% B over 5 min, to 40% B over 25 min, flow rate 60 mL/min. Seq ID 3 was isolated as an amorph freeze-dried solid (Y=2%), TFA salt. The peptide was solubilized at 1 mg/mL concentration in a mixture of acetonitrile/water 8/2: HCl 50 mM (10 eq) was added, and mixture stirred for 1 hour at room temperature. After lyophilization, the peptide was dissolved in acetonitrile/water 8/2 and freeze dried again. The peptide was analyzed by LC/MS (Method C). [M+3H]³⁺ mass signal found under the peak with retention time 2.90 min revealed the peptide mass 1171.0 which is in line with the expected value of molecular weight 3509.17.

Procedure for the synthesis of Seq ID 32:
QHQMLFYKDDVLFYNISSMK-Ttds-Ttds-Ttds-Ttds-K(N₃)-
CONH₂

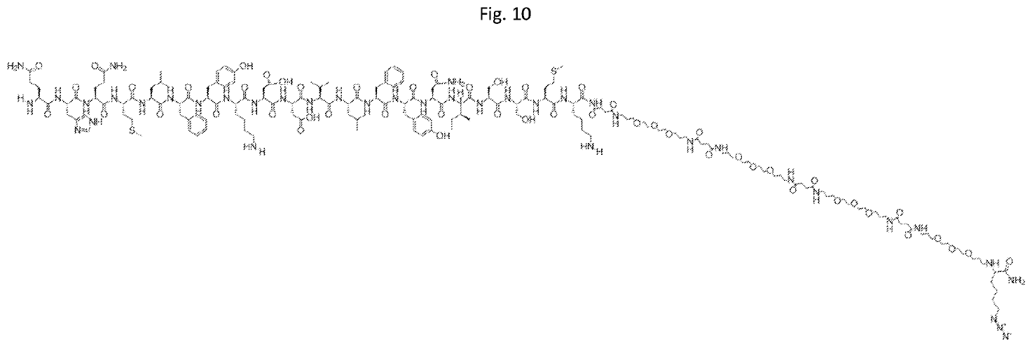

The structure of the peptide is shown in FIG. 10.
Seq ID 32 (PepSP1375) spans the same region Gln70-Lys89 of CD31 IgL1-A and a long linker of Ttds units.
During peptide assembly on solid phase, double acylation reactions were performed for Val and Leu. Fmoc deprotections were performed with a double treatment of the resin with 20% (V/V) piperidine in DMF for 120 at 90° C. up to the Asp residue in position 9: after this residue deprotections were performed at room temperature to minimize aspartimide formation. The cleavage was performed according to the general procedure using Mix 2. The mixture was analyzed by analytical UPLC and verified by ESI+ mass spectrometry and it was lyophilized to afford the crude title compound (Y=70%).
Purification of the crude material was performed using an XBridge C18 (250×50 mm, 120A, 10 μm) column and a gradient of Solvent B as follows: 15% B for 5 min; 15% of B to 35% in 20 min; flow rate: 80 mL/min. Product-containing fractions were collected and lyophilized to obtain the purified product as a TFA salt (Y=10%). The purified peptide was analyzed by LC/MS (Method B). [M+4H]⁴⁺ mass signal found under the peak with retention time 4.20 min revealed the peptide mass 969.2 which is in line with the expected value of molecular weight 3870.58.

Procedure for the synthesis of Seq ID 33:
hCHQMLFYKDDVLFYNISShCK-Ttds-Ttds-Ttds-Ttds-K(N₃)-
CONH₂

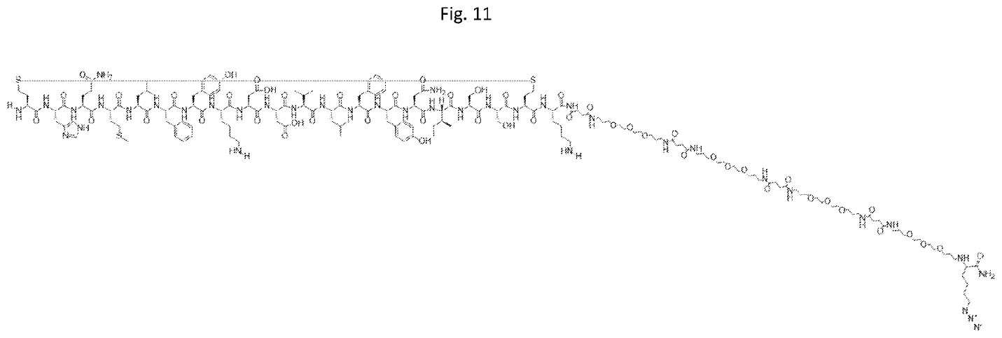

The structure of the peptide is shown in FIG. 11.
Seq ID 33 (PepSP1374) spans the same region Gln70-Lys89 of CD31 IgL1-A of Seq ID 32 with mutations to homocysteines: Gln70hCys and Met88hCys that results in a disulfide bond with a cyclic structure.
During peptide assembly, double acylation reactions were performed for Val and Leu. Fmoc deprotections were performed with a double treatment of the resin with 20% (V/V) piperidine in DMF for 120 seconds at 90° C. up to the Asp residue in position 9: after this residue deprotections were performed at room temperature to minimize aspartimide formation. The cleavage was performed according to the general procedure using Mix 2. The mixture was analyzed by analytical UPLC and verified by ESI+ mass spectrometry and it was lyophilized to afford the crude title compound (Y=70%). Crude peptide was analyzed by analytical UPLC and verified by ESI+ mass spectrometry. Crude material was dissolved in a 9/1 mixture of DMSO/H₂O at 1 mg/mL concentration: aqueous NH₃ added to reach pH 9 and the mixture was stirred for 48 hours at room temperature. After this time the formation of the disulfide bridge was confirmed by UPLC and the mixture was freeze-dried. Purification of the crude material was performed with a Waters XBridge C18 (250×50 mm, 120A, 10 μm) column using the following gradient: 15% B for 5 min; 15% of B to 35% in 20 min; flow rate 80 mL/min. Product-containing fractions were collected and lyophilized to obtain the purified product as a TFA salt (Y=10%). The purified peptide was analyzed by LC/MS (Method B). [M+4H]⁴⁺ mass signal found under the peak with retention time 4.05 min revealed the peptide mass 962.4 which is in line with the expected value of molecular weight 3843.58.

Procedure for the synthesis of Seq ID 34:
YKSTVIVNNKEKTTAE-PEG4-K(N₃)-CONH₂

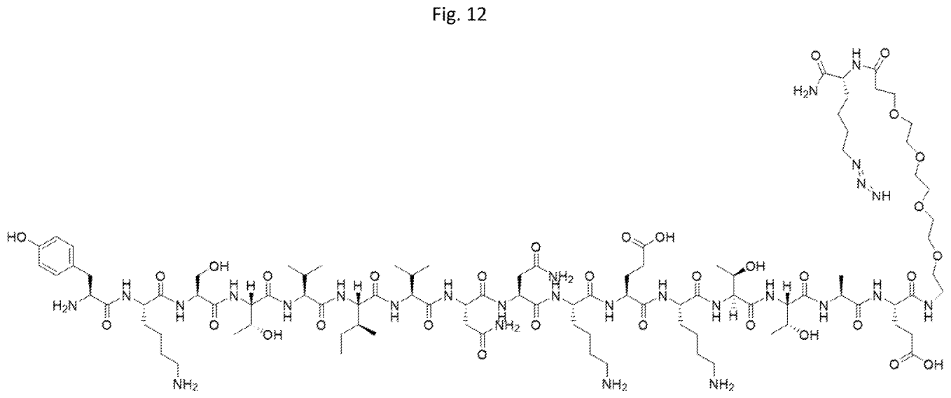

The structure of the peptide is shown in FIG. 12.
Seq ID 34 (PepSP1072) spans the region Tyr107-Glu122 of CD31 IgL1-A.
During the assembly, double acylation reactions were performed for all natural amino acids and Fmoc-NH-PEG4-COOH. After the synthesis and the cleavage performed according to the general procedure (Mix 1), the mixture was analyzed by analytical UPLC and verified by ESI+ mass spectrometry; lyophilized to afford the crude title compound (Y=70%). LCMS anal. calc. for C96H166N27033: 2225.21 Da; found; 1114.0 (M+2) 2+. The crude peptide was purified by reverse-phase HPLC using preparative Waters XBridge C18 (250×50 mm, 120A, 10 μm) column and the following gradient of eluent B: 5% B to 5% B over 5 min, 5% B to 25% B over 20 min, flow rate 80 mL/min. Seq ID 34 was isolated as an amorph freeze-dried solid (Y=25%) as TFA salt. The purified peptide was analyzed by LC/MS (Method A). [M+3H]³⁺ mass signal found under the peak with retention time 2.97 min revealed the peptide mass 743.0 which is in line with the expected value of molecular weight 2226.54.

Procedure for the synthesis of Seq ID 23:
hCYKSTVIVNNKEKTTAEYhC-(Ttds)₄-K(N₃)-NH₂

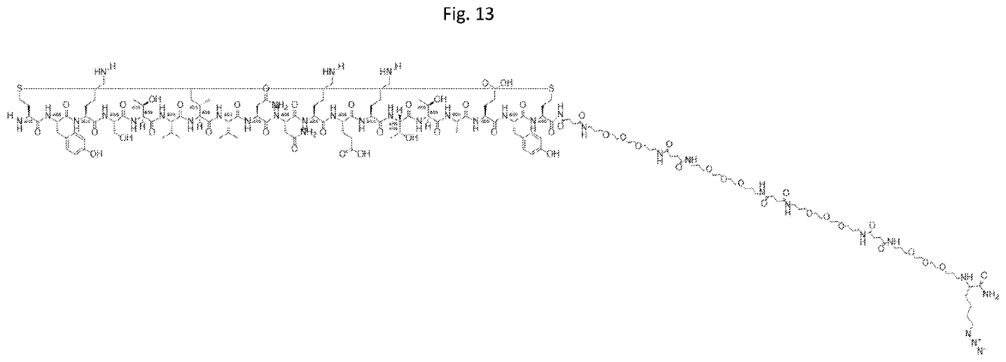

The structure of the peptide is shown in FIG. 13.
Seq ID 23 (PepSP1376) spans the region 106-124 where position 106 and 124 are mutated to Homocysteines that results in a disulfide bond with a cyclic structure. Synthesis and cleavage were performed according to the general procedure (Mix 1); cleaved the mixture was analyzed by analytical UPLC and verified by ESI+ mass spectrometry and it was lyophilized to afford the crude title compound (Y=65%). Crude material was dissolved in a 9/1 mixture of DMSO/H₂O at 1 mg/mL concentration: aqueous NH₃ added to reach pH 9 and the mixture was stirred for 48 hours at room temperature. The formation of the disulfide bridge was confirmed by UPLC and the mixture was freeze-dried. The crude peptide was purified by reverse-phase HPLC using XBridge C18 (250×50 mm, 120A, 10 μm) column and the following gradient of eluent B: 5% B to 5% B over 5 min, to 25% B over 20 min, flow rate 80 mL/min. Product-containing fractions were collected and lyophilized to obtain the purified product as a TFA salt (Y=28%). The purified peptide was analyzed by LC/MS (Method A). [M+3H]³⁺ mass signal found under the peak with retention time 3.57 min revealed the peptide mass 1195.4 which is in line with the expected value of molecular weight 3583.22.

Procedure for the synthesis of Seq ID 35:
K(N₃)-PEG4-PRVTLDKKEAIQGGIVRVNSSVPEEK-CONH₂

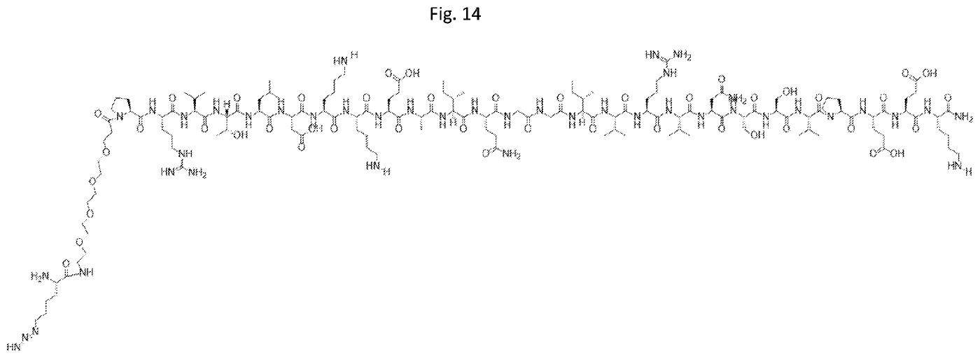

The structure of the peptide is shown in FIG. 14.
Seq ID 35 (PepSP1071) spans the region Pro133-Lys158 of CD31 IgL2-A.
Synthesis and cleavage performed according to the general procedure (Mix 1); the cleaved mixture was analyzed by analytical UPLC and verified by ESI+ mass spectrometry and it was lyophilized to afford the crude title compound (Y=54%). The crude peptide was purified by reverse-phase HPLC using preparative double Waters DeltaPack C18 cartridges (100×40 mm, 300 Å, 15 μm). The following gradient of eluent B was used: 5% B to 5% B over 5 min, to 25% B over 20 min, flow rate 80 mL/min. Product-containing fractions were collected and lyophilized to obtain the purified product as a TFA salt (Y=35%). The purified peptide was analyzed by LC/MS (Method A). [M+3H]³⁺ mass signal found under the peak with retention time 3.57 min revealed the peptide mass 1085.0 which is in line with the expected value of molecular weight 3251.76.

Procedure for the synthesis of Seq ID 36:
Ac-K(N₃)-(Ttds)₄-PRhCTLDKKEAIQGGIVRVNhCSVPEEK-
CONH₂

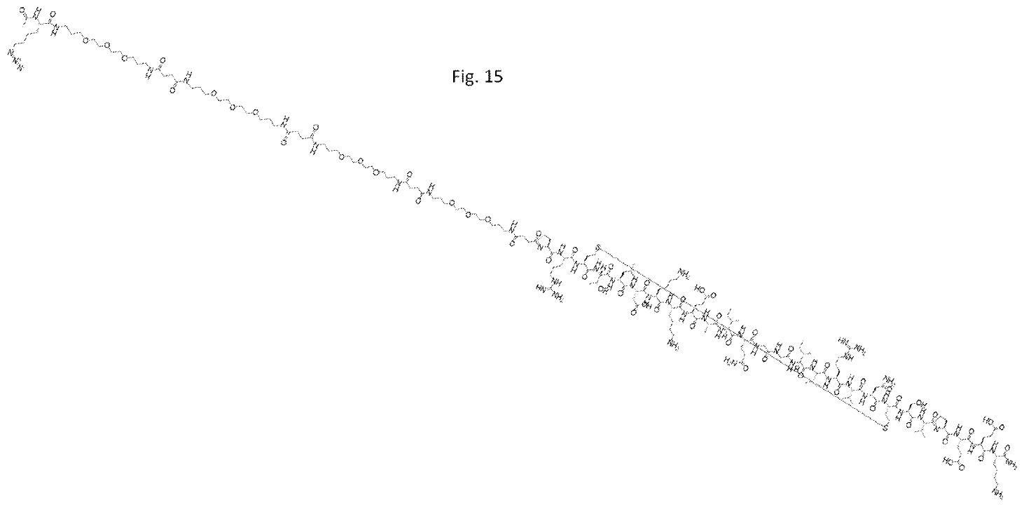

The structure of the peptide is shown in FIG. 15.
Seq ID 36 (PepSP1380) spans the region Pro133-Lys158 of CD31 IgL2-A as seq ID 35 but with mutations homocysteines at positions 135 and 152 that results in a disulfide bond with a cyclic structure.
Synthesis performed according to the general procedure. During peptide assembly, double acylation reactions were performed for the residues in the fragments Ser-Val-Pro, Ile-Val-Arg-Val and Pro-Arg-homoCys-Thr-Leu. Fmoc deprotections were performed with a double treatment of the resin with 20% (V/V) piperidine in DMF for 120 seconds at 90° C. up to Asp in position 6: after this residue deprotections were performed at room temperature to minimize aspartimide formation. The four Ttds and the terminal Lys-azide residues were introduced manually using HOAT (5 eq) and DIPC (5 eq) as coupling reagents in DMF: triple coupling of the last Ttds residue was performed to afford a full conversion. At the end of the assembly the resin was treated with Ac₂O (10 eq) in DMF for 30 mins. The cleavage was performed according to the general procedure using Mix 1, the mixture was analyzed by analytical UPLC and verified by ESI+ mass spectrometry and it was lyophilized to afford the crude title compound (Y=73%). Crude material was dissolved in a 9/1 mixture of DMSO/H₂O at 1 mg/mL concentration: aqueous NH₃ added to reach pH 9 and the mixture was stirred for 72 hours at room temperature. After this time the formation of the disulfide bridge was confirmed by UPLC, TFA added to quench the reaction mixture and then it was freeze dried. The crude peptide was purified by RP-HPLC using Reprosil C8 (250×40 mm, 200 Å, 5 μm) column and the following gradient of eluent B was used: 20% B for 5 min; 20% of B to 40% in 25 min; flow rate 60 mL/min. Product-containing fractions were collected and lyophilized to obtain the purified product as a TFA salt (Y=5%). The purified peptide was analyzed by LC/MS (Method B). [M+4H]⁴⁺ mass signal found under the peak with retention time 3.23 min revealed the peptide mass 1076.8 which is in line with the expected value of 4301.09 molecular weight.

Synthesis of Seq ID 37:
[#CH₂CONH₂GTYKSTVIVNNKEKTTAEYQ-(Ttds)₄-K(N₃)-NH₂]-
[QHQMLFYhC^{#}DDVLFYNISSMK-CONH₂]

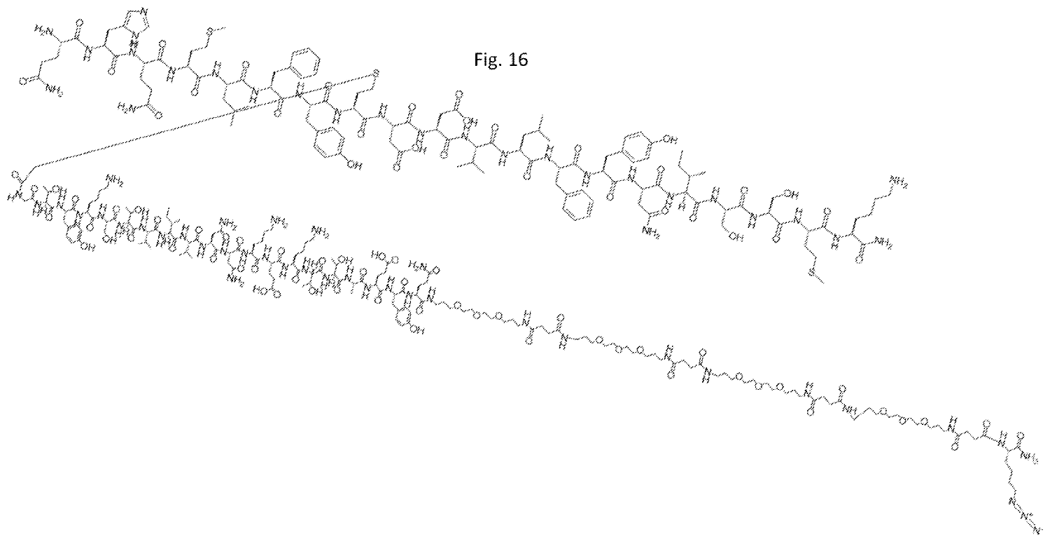

The structure of the peptide is shown in FIG. 16.
Seq ID 37-38 (SP1379) is the heterodimer obtained through a thioether linkage between hCys8 of intermediate A (INT A) (QHQMLFYhC#DDVLFYNISSMK-CONH2) and the bromoacetyl group of intermediate B (INT B) [BrCH₂CO-G#TYKSTVIVNNKEKTTAEYQ-(Ttds)4-K(N₃)—NH₂].
A 5 mM solution of INT B (1 eq) in DMSO was added dropwise to a 2 mM solution of INT A (1 eq) in DMSO in the presence of DIPEA (5 eq). The reaction was stirred at room temperature 1 hour during which time reaction progression was monitored by LCMS. TFA was added to reaction mixture to reach pH 4 and crude purified by RP-HPLC using Reprosil C8 (250×40 mm, 200 Å, 5 μm) column. The following gradient was employed: 25% B for 5 min; 25% of B to 45% in 25 min; flow rate: flow rate 40 mL/min. Product-containing fractions were collected and lyophilized to obtain the purified product as a TFA salt (Y=21%). The purified peptide was analyzed by LC/MS (Method B). [M+4H]⁴⁺ mass signal found under the peak with retention time 4.35 min revealed the peptide mass 1545.5 which is in line with the expected value of molecular weight 6173.13.

Synthesis of Seq ID 38:
[Ac-K(N₃)-(Ttds)₄-PRVTLDKKEAIQGGIVRVNSSVPEEK-Ttds-
K^{#} (CH₂CO)-CONH₂]-[QHQMLFYhC^{#}DDVLFYNISSMK-CONH₂]

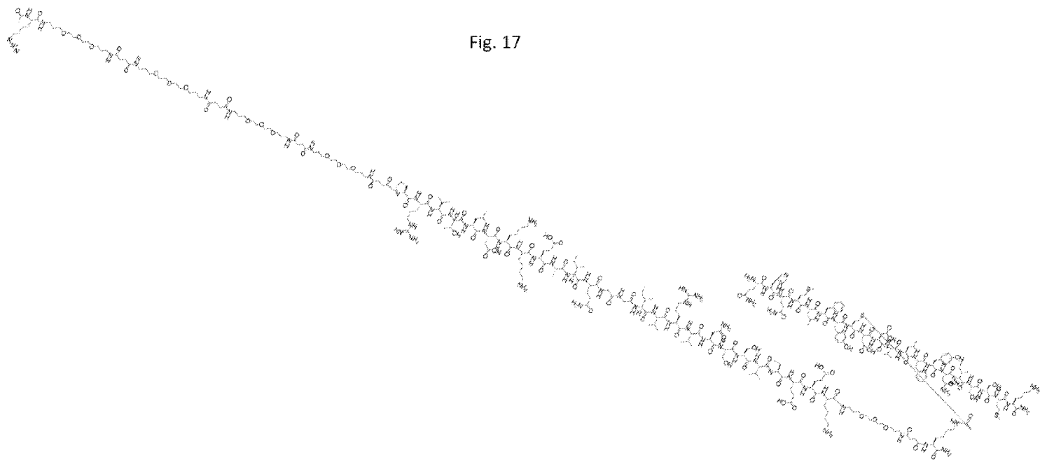

The structure of the peptide is shown in FIG. 17.
Seq ID 38 is the heterodimer obtained through a thioether linkage between hCys8 of intermediate A (INT A) (QHQMLFYhCDDVLFYNISSMK-CONH₂) and the bromoacetyl group of intermediate C (INT C) [Ac—K(N₃)-(Ttds)₄-PRVTLDKKEAIQGGIVRVNSSVPEEK-Ttds-K (BrCH₂CO)—CONH₂].
A 5 mM solution of INT C (1 eq) in DMSO was added dropwise to a 2 mM solution of INT A (1 eq) in DMSO in the presence of DIPEA (5 eq). The reaction was stirred at room temperature 1 hour during which time reaction progression was monitored by LCMS. TFA was added to reaction mixture to reach pH 4 and crude purified by RP-HPLC using Reprosil C8 (250×40 mm, 200 Å, 5 μm) as column. The following gradient of eluent B was employed: 25% B for 5 min; 25% of B to 45% in 25 min; flow rate: flow rate 40 mL/min. Product-containing fractions were collected and lyophilized to obtain the purified product as a TFA salt (Y=5%). The purified peptide was analyzed by LC/MS (Method B). [M+5H] 5+ mass signal found under the peak with retention time 4.59 min revealed the peptide mass 1445.9 which is in line with the expected value of 7221.47 molecular weight.

Synthesis of Seq ID 1:
INT A: QHQMLFYhCDDVLFYNISSMK-CONH₂

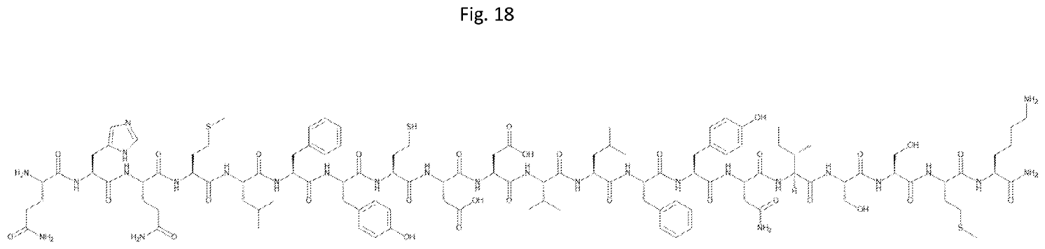

The structure of the intermediate A is shown in FIG. 18.
The linear precursor common intermediate for heterodimers SEQ ID no. 37 (SP1379) and SEQ ID no. 38 (SP1383) was prepared following the general procedure for the synthesis and cleavage: all the Fmoc deprotections were performed at room temperature. The crude peptide was purified using Reprosil C4 (250×40 mm, 120 Å, 5 μm) as column and the following gradient of eluent B: 20% B for 5 min; 20% of B to 40% in 25 min; flow rate: flow rate 60 mL/min, wavelength 214 nm. Product-containing fractions were collected and lyophilized to obtain the purified product as a TFA salt (Y=2%).

INT B:
Synthesis of Seq ID 2:
(BrCH₂CO-GTYKSTVIVNNKEKTTAEYQ-(Ttds)₄-K(N₃)-NH₂)

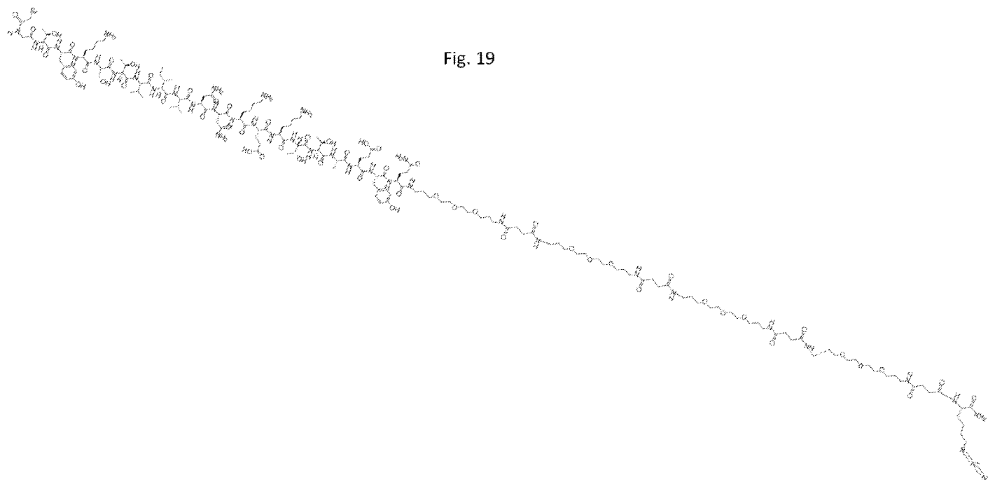

The structure of the intermediate B is shown in FIG. 19.
Linear precursor for heterodimer SEQ ID no. 37-38 (SP1379), was prepared following the general procedure for the synthesis. At the end of the assembly, the free amino group on the last residue on the resin (gly) was treated with bromoacetic acid (5 eq) according to the DIC/HOAt method (4 equivalent excess with respect to resin loading) in DMF. The mixture was shaken at room temperature for 1 h and the reaction was monitored by Kaiser Test. The resin was filtered and washed with DMF/DCM/DMF (6/6/6 time each) and treated with the cleavage mixture (Mix 1) as reported in the general synthetic procedure. The crude peptide was purified by RP-HPLC using Reprosil C4 (250×40 mm, 120 Å, 5 μm) column and the following gradient: 20% B for 5 min; 20% of B to 40% in 25 min; flow rate: flow rate 60 mL/min, wavelength 214 nm. Product-containing fractions were collected and lyophilized to obtain the purified product as a TFA salt (Y=30%).

INT C:
Synthesis of Seq ID 3:
Ac-K(N₃)-(Ttds)₄-PRVTLDKKEAIQGGIVRVNSSVPEEK-Ttds-
K(BrCH₂CO)-CONH₂

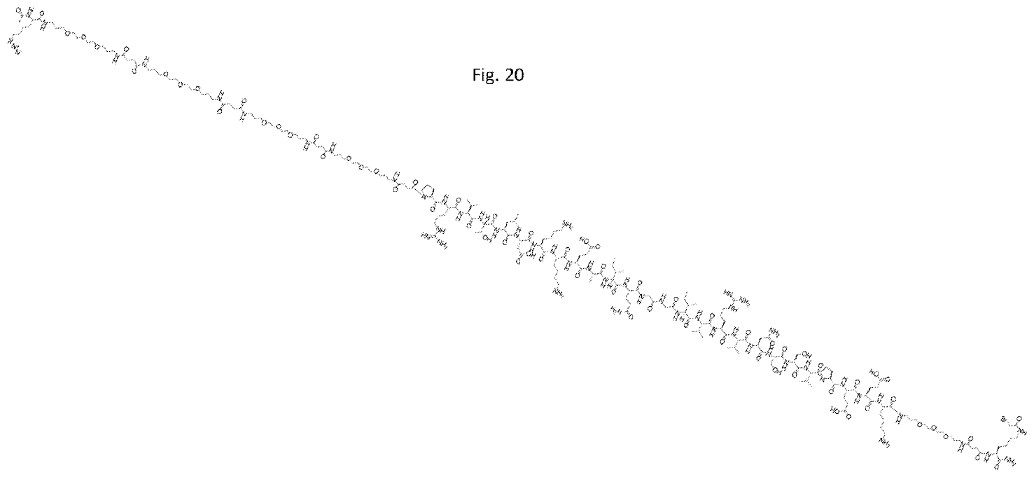

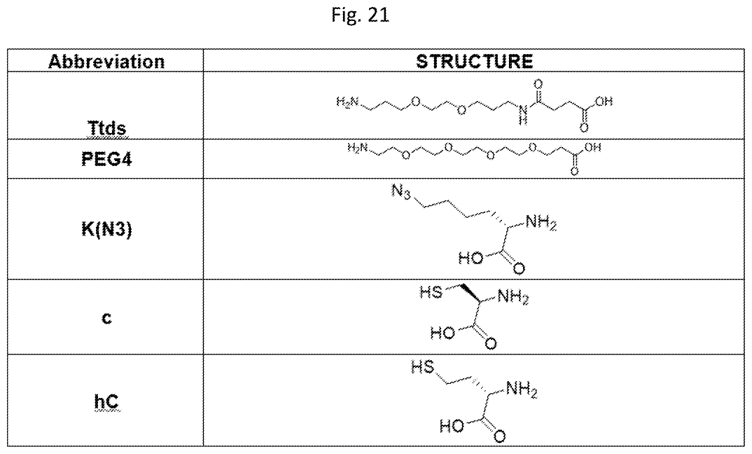

The structure of the intermediate C is shown in FIG. 20.
Linear precursor C for heterodimer SEQ ID no. 39-40 (SP1383) was prepared following the general synthetic procedure. The first amino acid loaded onto the resin was Lys protected with alloc on the side chain amino group. Double acylations were performed on the following residues: SVP, IVRV and PRVTL: after the PRVTL residues all Fmoc deprotections were performed at room temperature.
At the end of the assembly the resin was suspended in DCM: phenylsilane (24 eq) and Pd (PPh3) 4 (0.25 eq) were added and the resin was shaken for 30 minutes. The cycle was repeated twice. At the end of the second cycle a solution of 0.5% DIPEA and 0.5% Sodium dithiocarbamate in DMF is percolated to the resin to remove Palladium residues. Coupling of the Lys deprotected amino group with bromoacetic acid (5 eq) was performed with DIC/HOAt method (4 equivalent excess with respect to resin loading) in DMF. The mixture was shaken at room temperature for 1 h and the reaction was monitored by Kaiser Test. The resin was filtered and washed with DMF/DCM/DMF (6/6/6 time each) and treated with the cleavage mixture (Mix 1) as reported in the general synthetic procedure. The crude peptide was purified: Reprosil C4 (250×40 mm, 120 Å, 5 μm) column and the following gradient: 20% B for 5 min; 20% of B to 40% in 25 min; flow rate: flow rate 60 mL/min, wavelength 214 nm. Product-containing fractions were collected and lyophilized to obtain the purified product as a TFA salt (Y=30%).

(SP547)
Synthesis of Seq ID 41:
Ac-K(N₃)-GGSGGSGG-YKDDVLFYNISSMKST-NH₂

Seq ID 41 (PepSP547) is a linear peptide spanning region Tyr76-Thr91 of CD31 IgL1-A with the azide-Linker position at the N terminus.

(SP548)
Synthesis of Seq ID 42:
Ac-YKDDVLFYNISSMKST-GGSGGSGG-K(N₃)-NH₂

Seq ID 42 (PepSP548) is also a linear peptide spanning the same region Tyr76-Th91 of CD31 IgL1-A but the azide-Linker position at the C-terminus

### Protocol

Nitinol disks are left untouched (bare metal) or coated with a domain 1, domain 2 or domain 1 and 2 CD31 analog peptide. The tested peptide is grafted to the nitinol disk following a polydopamine, DBCO-PEG4-amine and azide CD31 derivative peptide using a sequential dip-coating layering procedure. After this step, all the disks are incubated with Human aortic endothelial cells for 48h. Disks are then rinsed and the cells are fixed in a formalin solution before staining. The staining is fulfilled using phalloidin coupled to AlexaFluor®488 dye (green fluorescence for Actin), DAPI is used for DNA staining and a domain 1 CD31 antibody coupled to APC is used for external Domain 1 staining. The cell adhesion and Actin/CD31 expression was evaluated by image analysis of the surface after staining.

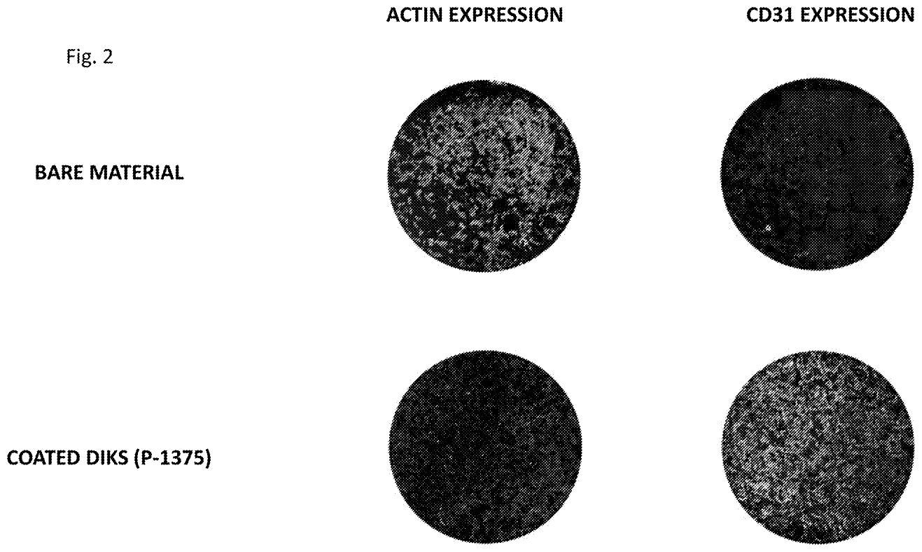

The results are shown in FIGS. 2 through to 6.

In particular, as per FIG. 2 the bare or coated nitinol disks were placed at the bottom of culture wells. Primary Human Aortic Endothelial Cells (HAEC) were allowed to grow on the disks for a period of 48 h. The disks were thereafter rinsed with phosphate buffered saline, fixed in formalin, and processed for fluorescence microscopy. Disk-adherent cells were immersed in a solution comprising Hoechst 33342 (1 μg/ml, to stain the nuclei), phalloidin coupled to AlexaFluor®488 dye (0.5 μM, to stain F-actin fibers) and a monoclonal antibody directed against CD31 domain 1, coupled to allophycocyanin (to detect the presence and localization of intact CD31 at the cell surface) for 1 hour at 37° C., washed and mounted face down on glass bottom imaging chambers for imaging with an inverted fluorescence microscope. Representative images show that HAEC growing on bare material displayed a strong F-actin staining, reflecting enhanced formation of stress fibers whereas the expression of intact CD31 was rather faint. At the opposite, disks coated with CD31-mimetic peptides displayed little stress fibers signal and an intense and uniform CD31 expression at the cell borders, reflecting a more physiologic endothelial phenotype.

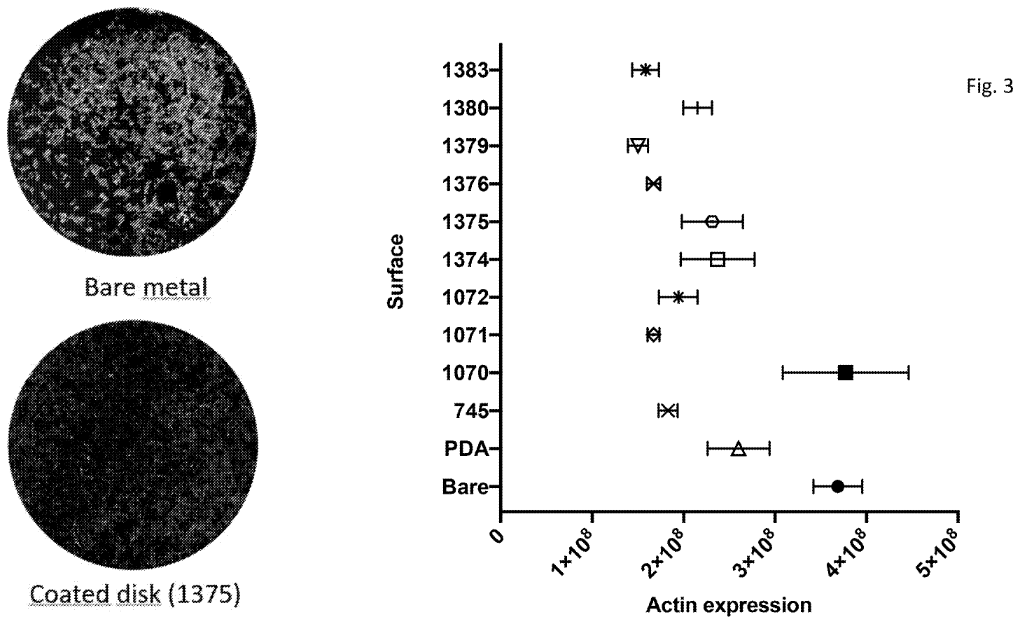

FIG. 3 shows quantitative analysis of the signal on individual disk images is expressed as “Integrated Density” (signal Density multiplied by the area of positive staining, as reported by ImageJ analysis open software). Surface type is indicated on the Y axis, data are expressed as average±SD (N=6/surface type). Each dot corresponds to a specific surface modification. The F-actin expression relates the cellular stress of the cell in presence of the exogenous material. All surface modification lead to a decrease in cellular stress compared to the bare metal, except for the SP1070 peptide, which does not show an influence on the cellular stress compared to the bare metal.

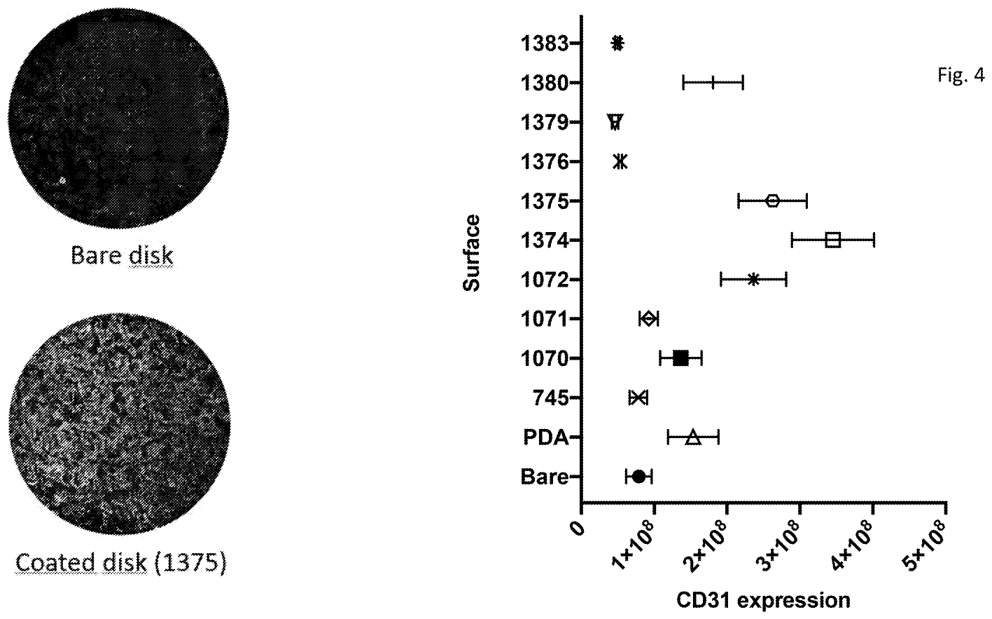

In FIG. 4 the surface type is indicated on the Y axis. Each dot corresponds to a specific surface modification. Data are expressed as “Integrated Density” average±SD (N=6/surface type). CD31 expression relates the capacity of endothelial cells to adopt a physiologic phenotype while growing on a given substrate. An important variability is observed in terms of CD31 expression by HAEC according to the surface modification of the nitinol disks. Four of the displayed CD31 analog peptides (peptides SP 1072-1374-1375 and 1380) exert an evident positive effect in terms of CD31 expression as compared to the other modified disks and bare controls.

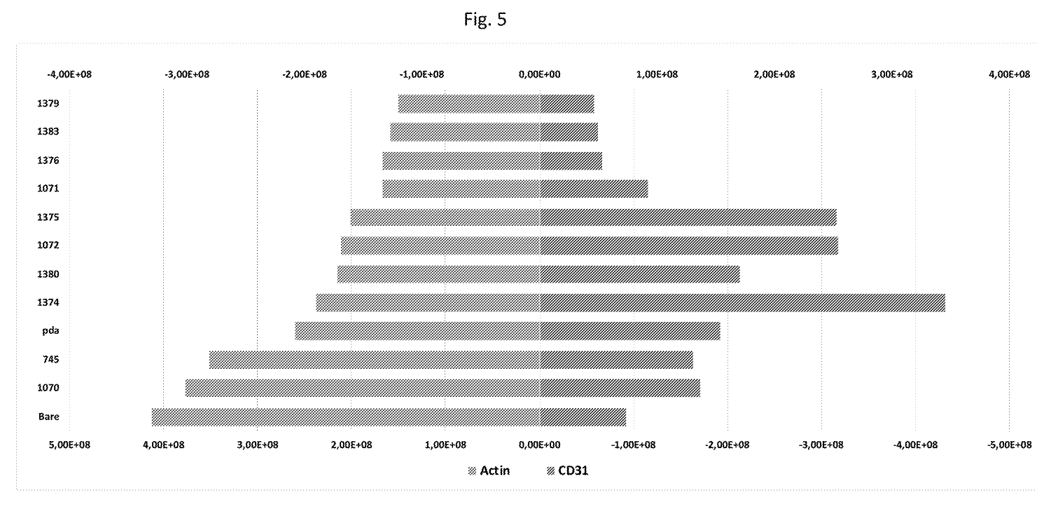

The “mirror” bars of FIG. 5 report the average Integrated Density of F-actin (left) and of CD31 (right) staining for each of the indicated (Y axis) type of peptide coated onto the disk surface and bare disks. Data are sorted (from the top to the bottom) according to the increase in the cellular stress (actin expression) of each coating. As shown on the figure, the degree of cellular stress (as detected by a high actin expression) and impaired endothelial phenotype (documented by a low CD31 expression) is evident on HAEC grown onto bare metal disks. On the contrary, the CD31-mimetic surfaces, such the disks coated with the SP 1374 peptide, lead to a low cellular stress (low actin expression) and consistent functional endothelial phenotype (high CD31 expression).

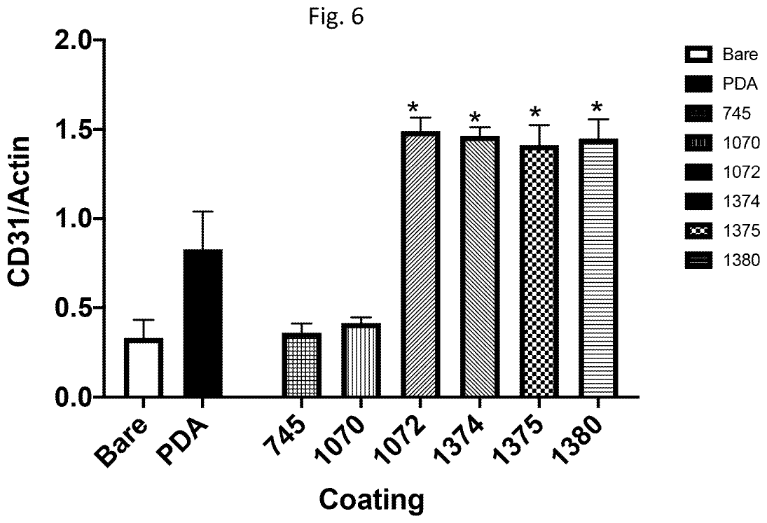

The data shown in FIG. 6 are expressed as the ratio between CD31 and F-actin expression. As compared to bare metal and disks coated with PDA only, cells growing onto surfaces coated with peptides SP 1072, 1074, 1075 and 1080 clearly show a higher CD31/actin ratio, reflecting a more physiological endothelial cell phenotype.

### Example 1

### Functional Score and Biomimetic Performance

### Functional Score

The effect of different peptides, belonging to the different groups, were tested in batches following the same protocol as in FIG. 6. Different peptides from each group were tested in batches and the effect of individual peptides was repeatedly assessed in separate experiments. Data from all experiments were obtained by computer-assisted analysis of images of the disk side covered with endothelial cells, captured in the blue, green and red channel of an inverted fluorescence microscope. The functional score of the peptides belonging to the different groups was calculated by multiplying the number of cells (as detected by the number of blue, Dapi+ nuclei) for the integrated density of CD31 expression (red signal provided by the binding of a mouse anti-human CD31 monoclonal antibody, clone 9G11, conjugated allophycocyanin) and their product being divided for the integrated density of the F-actin polymerization (Phalloidin coupled to a green fluorochrome).

The formula used for calculating the score using the raw data from all experiments was the following:

$$\frac{N ° of nuclei \times Intregrated Density of CD 31 staining}{Integrated density of F - actin staining}$$

The score was scaled 0-1 using the formula (x-min)/(max-min) in each experiment.

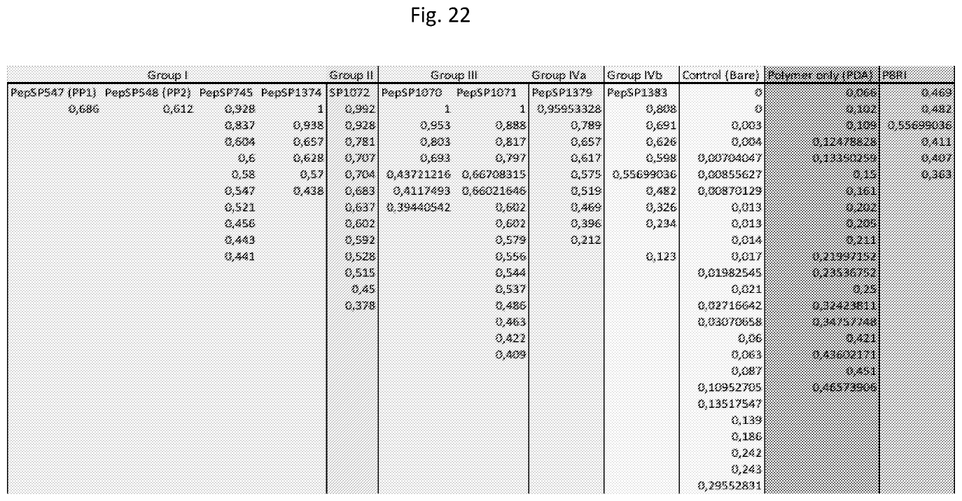

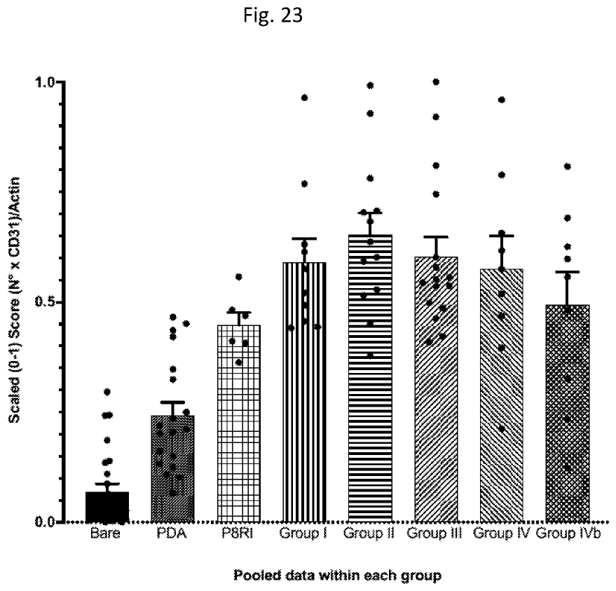

The data are reported in the table shown in FIG. 22 and in the graph of FIG. 23.

### Biomimetic Performance

The functional effect (biomimetic performance) of the peptides belonging to the different groups (peptide ID indicated in parentheses), was screened using the same protocol as described for the example in FIG. 6. Different peptides from each group were tested in batches and the effect of individual peptides was repeatedly assessed in separate experiments. Data from all experiments were obtained by computer-assisted analysis of images of the disk side covered with endothelial cells, captured in the blue, green and red channel of an inverted fluorescence microscope. The functional score of the peptides belonging to the different groups was calculated by multiplying the number of cells (as detected by the number of blue, Dapi+ nuclei) for the integrated density of CD31 expression (red signal provided by the binding of a mouse anti-human CD31 monoclonal antibody, clone 9G11, conjugated allophycocyanin) and their product being divided for the integrated density of the F-actin polymerization (Phalloidin coupled to a green fluorochrome).

The formula used for calculating the score using the raw data from all experiments was the following:

$$\frac{N ° of nuclei \times Intregrated Density of CD 31 staining}{Integrated density of F - actin staining}$$

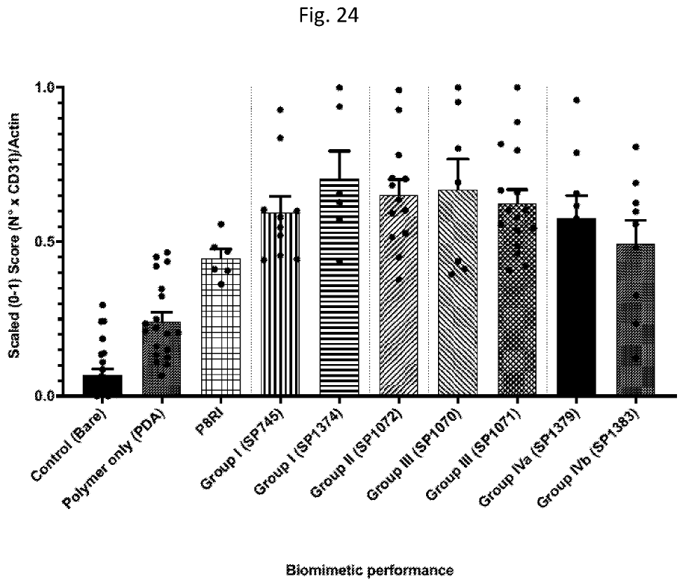

The score was scaled 0-1 using the formula (x-min)/(max-min) in each experiment and scaled scores from all experiments are shown in FIG. 24.

### Example 2

Further CD31/Actin Scores of Peptides According to the Invention and Comparison with Prior Art Peptide P8RI

### Materials and Methods

The same protocol as in Example 1 was followed, using peptides according to the invention (SP547, SP548 SP745, SP1374, SP1072, SP1070, SP1071, SP1379 and SP1383) as defined above and the prior art peptide P8RI of sequence KWPALFVR (SEQ ID NO: 52).

### Results

The “CD31/Actin” score, resulting from the integrated density of CD31 immunostaining signal divided by the signal of phalloidin, which binds to the polymerized F-actin of the cytoskeleton, has been used to identify the physiological/non-stressed character of the arterial endothelial cells growing onto the surface of experimental disks placed at the bottom of culture wells. A higher score indicates a more physiological status, while lower score reflects a stressed status of endothelial cells, associated with a pro-thrombotic/pro-inflammatory activity (as confirmed by the level of soluble PAI-1 and IL-6 in the supernatant of the individual culture wells). Therefore, the higher the CD31/Actin score, the better.

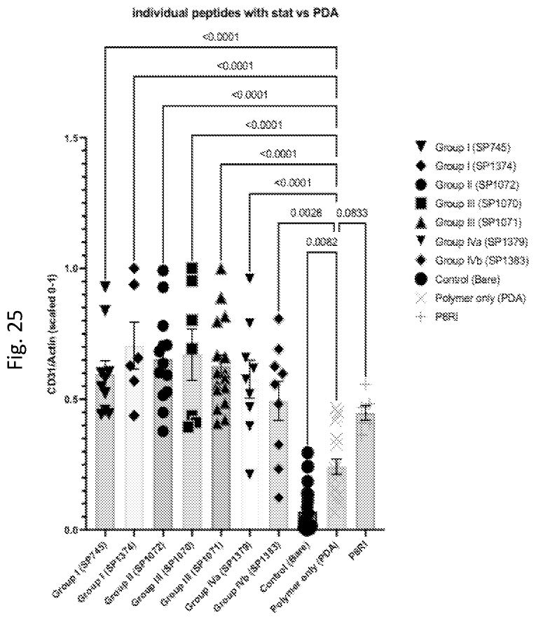

Result issued from repeated experiment performed with the different peptides are presented in FIG. 25. The CD31/Actin scores obtained with the new peptides SP745, SP1374, SP1072, SP1070, SP1071, SP1379 and SP1383 according to the invention are significantly higher than those obtained by the bare metal disks or by disks coated with PDA alone. While statistical significance cannot be achieved with only one measure, sections coated with new peptides SP547 and SP548 seem to have CD31/Actin scores equivalent to those of sections coated by other peptides according to the invention. In addition, the mean CD31/Actin scores of all peptides according to the invention are higher than the mean CD31/Actin score of prior art peptide P8RI.

### Conclusion

The above results confirm that disks coated with the new peptides according to the invention display higher and thus better CD31/Actin scores than sections coated with PDA only, and also better CD31/Actin scores than sections coated with prior art peptide P8RI.

### Example 3

### In Vivo Effect of Flow-Diverter Stents (CFD) Coated by Peptide SP1072 According to the Invention Compared to Stents Coated by Hydrophilic Polymer NTMA Only

### Materials and Methods

### Stents

Flow-diverter stents (CFD) are braided meshes made of nitinol, from Sinomed. They are coated by one of the following coatings:
1. SP1072 peptide according to the invention, or
2. Hydrophilic polymer eG NTMA (obtained from the electrografting of N-[Tris(hydroxymethyl)Methyl] Acrylamide).

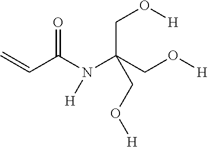

For SP1072-coated flow diverter stents, the coating was performed using the following materials and protocol:

### Materials:

### Demineralized Water

Tris 10 mM (MW: 121.135 i.e. 1.21 mg/mL) pH 8.5 buffer
0.6 g Trizma Base (CAS 77-86-1)
500 ml demineralized water
Adjust pH to 8.5 with HCl
Filter with 0.45 μm filter
Store at room temperature
Polydopamine (PDA) at 2 mg/mL
Dopamine hydrochloride 99% (CAS 62-31-7, MW: 189.64, store at 4° C.)
Solubilize the right amount of Dopamine hydrochloride in 10 mM Tris buffer pH 8.5 to obtain a final concentration of 2 mg/mL solution (keep in the dark)
DBCO (MW: 678.79, DBCO-sulfo-PEG (4)-NH₂ (ref: RL-2421, IRIS biotech)

Once the compound received, prepare stock weights of 15-20 mg. Solubilize the powder to create a stock solution at 20 mg/mL in 10 mM Tris buffer pH 8.5. Prepare the wanted volume for the coating process at a final concentration of 300 μM (203 μg/mL)

SP1072 peptide at 50 μg/mL.

### Protocol:

1) Transfer each stent in a 5 mL polypropylene round bottom tube (Bd Falcon Ref 352063). Insure the stent drops at the bottom of the tube.

2) Add 4.5 mL of a 2 mg/mL PDA solution per tube. Homogenize each tube on a rotary wheel (approx. 20 rpm) overnight (18+/−2h) at room temperature and protect from light. Ensure that the stent moves in the tube and does not stay stuck in the cap. The stent color should change from silver to brown/black. After the incubation period the solution will become black with small PDA aggregates

3) Transfer each stent in a new tube and rinse 3-times with demineralized water. homogenize on the rotary wheel 5 min at each rinse.

4) Leave the last rinse and sonicate the tube for at least 30s to discard any remaining PDA chunk.

5) Transfer each stent in a 2 mL-Eppendorf tube containing 1.8 mL of DBCO (300 μM) per tube. Homogenize each tube on a rotary wheel (approx. 20 rpm) overnight at room temperature and protect from light.

6 Transfer each stent in a new tube and rinse 3-times with demineralized water. Homogenize on the rotary well 5 min at each rinse.

7) Transfer each stent in a 2 mL-Eppendorf tube containing 1.8 mL of CD31 peptide (50 μg/mL) per tube. Homogenize each tube on a rotary wheel (approx. 20 rpm) for 2 hours at room temperature and protect from light.

8) Transfer each stent in a new tube and rinse 3-times with demineralized water. Homogenize on the rotary well 5 min at each rinse.

9) Soak each stent in absolute Ethanol for 5 seconds before letting them dry.

Comparative hydrophilic polymer eG NTMA-coated flow diverter stents were obtained via the following protocol:

(1) Cleaning pretreatment: The stent frames were sonicated in acetone, ethanol, and water for injection for 10 min, respectively.

(2) Electro-grafting solution preparation: N-[tris(hydroxymethyl)methyl] acrylamide, concentration 0.30 M; NaNO₃, concentration 0.05 M; 4-nitrophenyl tetrafluoroborate diazonium salt, 0.005 M; the remaining amount is DMSO solvent.

(3) Electro-grafting process: the pretreated stents were used as the working electrode, and a platinum foil was used as the counter electrode, which was submerged in the electro-grafting solution, and a linear sweep voltage between −0.1 and −3.0 V and back was applied between the two electrodes with a voltage scan rate of 0.05 V/s for 10 cycles.

(4) Coating smoothing and drying: the electrografted scaffold was smoothed in acetone (2 L) with nitrogen bubbling (rate 0.5-10 L/min) for 10 min and placed in a vacuum drying oven at 40° C. for 2 h.

The same protocol is applied to a coupon with the same composition as the braided meshes. A film of 125 nm is obtained, as measured by profilometry (KLA-Tencor) on the step of a scratch done with a wooden stick on the electrografted coupon.

### Animal Procedures

A total of 8 healthy New Zealand White rabbits (including 2 spare animals), male and female, weighing about 3 kg, were included in this study. A total of 3 observation time points of 7 days, 30 days and 60 days postoperatively were set. The experiment was divided into two steps: 1) aneurysm animal modeling; 2) implantation of a dense mesh stent in the aneurysm-carrying artery and abdominal aorta for each animal. The stent implantation in the aneurysm-carrying artery was to be used to examine the safety and effectiveness of the stent in treating aneurysms, and the stent implantation in the abdominal aorta, covering a pair of lumbar arteries, was to be used to examine the safety of the tested stent. See Table 1 below for details.

TABLE 2
Experimental design

| Observation | N. of | N. of | Stent | |
|---|---|---|---|---|
| period | animals | brackets | implantation | |
| (days) | (only) | (pcs) | Part | Evaluation Indicators |
| 7 | 2 | 4 | Aneurysm- | 1. Routine blood and blood biochemical |
| | | | carrying | tests during the adaptation period, |
| | | | artery, | preoperatively, and prior to sampling. |
| 30 | 2 + 2 | 4 + 4 | abdominal | 2. Evaluate the operability and |
| | | | aorta | effectiveness of the dense mesh support. |
| 60 | 2 + 2 | 4 + 4 | | 3. The animals were kept until the |
| | | | | expected endpoint after the interven- |
| | | | | tional procedure, euthanized after |
| | | | | endpoint follow-up, and gross dissection |
| | | | | and tissue sampling were performed. |
| | | | | 4. Histopathological evaluation: |
| | | | | qualitative and quantitative analysis |
| | | | | of aneurysm-carrying arteries. |
| | | | | 5. SEM analysis; 7 D time point selected |
| | | | | abdominal aorta for SEM analysis; 30 D |
| | | | | time point selected abdominal aorta for |
| | | | | SEM analysis, carrier artery SEM detection |
| | | | | to be determined; 60 D time point selected |
| | | | | one carrier artery and one abdominal aorta |
| | | | | each for SEM analysis. |

For steps 1) and 2, aesthesia was performed as follows:

All surgical operations were performed using aseptic techniques, and the experimental animals were under general anesthesia during the implantation procedure.

On the day of surgery, the experimental animals are sedated and anesthesia is induced by intramuscular injection of 6 mg/kg of Sutex® 50. If needed, experimental animals can be anesthetized with isoflurane by inhalation using a breathing mask.

After successful induction of anesthesia, the experimental animal is connected to a ventilator device after being intubated through the oral plain view trachea to establish respiratory access and allow continuous inhalation of a mixture of anesthetic and oxygen to maintain anesthesia. It may be necessary to administer some atropine to the experimental animal to stop vomiting before the operation to prevent the animal from asphyxiation due to vomitus.

The anesthetized intubated experimental animal was placed on the operating table in the lateral or supine position, the animal was bound using a restraint band, and the position was photographed and recorded (to facilitate the same position and angle used for later observation). The surgical area of the right hind limb is prepared, disinfected, and sheeted. If the position of the animal is changed, a new sheet and preparation of the surgical site is required.

An intravenous needle is also placed in one of the peripheral venous vessels and medications or rehydration fluids are administered through the catheter as needed.

Step 1) (aneurysm animal modeling) was performed as follows:

(a) The rabbit is placed supine on the operating table and the neck is shaved. Routinely disinfect with iodophor and alcohol and then lay the towel.

(b) Locate the right common carotid artery: Take a median neck incision (1.5 cm above and below the superior sternal fossa) to cut the skin, separate along the lateral side of the right sternocleidomastoideole, find the right common carotid artery and free it, paying attention to vagus nerve protection to prevent the rabbit's heartbeat and respiration from slowing down or stopping. Every 20 minutes, saline drips were injected into the rabbit's cervical vessels and vagus nerve to keep them moistened. Two No. 1 silk wires were wrapped around the right common carotid artery.

(c) Fully expose the beginning segment of the right common carotid artery: open part of the right pectoral muscle with tissue shears, separate along the right common carotid artery proximally, and carefully separate the right common carotid artery and part of the right subclavian artery.

(d) Production of a closed lumen at the beginning of the right common carotid artery: a No. 1 wire is ligated at about 2.5 cm from the beginning of the right common carotid artery, another No. 1 wire is wrapped around a knot but not ligated, and the aneurysm clip is clamped at the beginning of the right common carotid artery near the right subclavian artery, ensuring that the inner side of the aneurysm clip is below the junction of the right common carotid artery and the right subclavian artery.

(e) Injection of elastase: Ophthalmic scissors cut the lateral wall of the artery at 1.5 cm from the beginning of the right common carotid artery, insert a cannula needle (22G cannula needle with a syringe at the end) containing elastase, ensure that the head of the needle is as close as possible to the aneurysm clip, ligate the cannula needle insertion with a silk thread to ensure that the fluid does not leak out, and inject approximately 75 U of porcine pancreatic elastase into the lumen of the tube.

(f) Vascular ligation: remove the trocar needle after 20 minutes of elastase ablation, ligate the puncture port, carefully loosen the aneurysm clip, and moisten it with a saline drip if necessary.

(g) Suture the wound: gauze dipped in dried accumulated blood, suture the muscle and skin layer by layer, and disinfect the incision with iodophor.

(h) Immediately after surgery, heparin sodium 200 U/kg and ceftriaxone sodium solution 0.3 g/kg were injected intravenously, and the animals were closely observed for vital signs until cleaning and fed in separate cages. Continue to use antibiotics for 3-5 days after surgery.

Step 2) (implantation of a dense mesh stent in the aneurysm-carrying artery and abdominal aorta for each animal) was performed as follows:

(a) Anesthesia: As described above.

(b) The right femoral artery was incised and a 5F protective vascular puncture sheath was inserted.

(c) A 5F introducer catheter was placed in the aortic arch and DSA angiography was performed.

(d) Placement of microcatheter: intravenous heparin was treated with systemic heparinization and a microcatheter was placed, which was pushed through a microguide wire from the subclavian artery to the distal end of the vessel and withdrawn.

(e) Stent implantation in the carrier artery position: The dense mesh stent system is inserted into the microcatheter through the introducer sheath, the push rod is pushed forward to the appropriate position, the introducer sheath is withdrawn, the push rod continues to be pushed until the stent is pushed into the carrier artery, and the stent position is adjusted so that the neck of the tumor is near the middle of the stent. Fix the push rod, retract the microcatheter and start to partially release the stent to the retrieval point position. If the position of the stent is satisfactory, continue to retract the microcatheter to release the stent completely; if the position is not satisfactory, push the microcatheter backwards to retrieve the stent into the microcatheter and adjust the position to release it again to completely cover the aneurysm neck. After stent placement is completed, withdraw the delivery system and microcatheter.

(f) Stent implantation in the abdominal aorta: After the release of the test article stent in the position of the carrier artery is completed, the guide catheter is retracted and placed in the position of the abdominal aorta, and a second stent is placed in the same way; the stent should be placed in the abdominal aorta through at least the beginning of a pair of lumbar arteries. After release is complete, the delivery system, microcatheter, and guide catheter are withdrawn and the femoral artery incision is sutured.

(g) Wait for the animal to wake up and feed it normally.

The test group was implanted with a dense mesh stent system in the aneurysm location (common carotid artery) and in the abdominal aorta of the animal model, and the control group was implanted with a dense mesh stent system in the aneurysm-carrying artery, where the abdominal aorta was implanted with the device to evaluate the effect of the stent on the penetrating and branch vessels.

In addition to heparin sodium injection 150 U/kg by IV during implantation, antiplatelet therapy (aspirin and clopidogrel) was given to the animals at 5 mg/kg each by feeding once a day for 3 days before surgery and at 5 mg/kg each by feeding postoperative to endpoint.

### Histopathological Analysis

The collected tissues of the stented segments of the carrier arteries were preserved in 10% neutral formalin for more than 48 hours of immersion fixation, dehydrated in alcohol gradient and treated with xylene transparency for histopathological analysis.

The resin-embedded stented segment of the aneurysm-carrying artery was sectioned transversely through the neck of the aneurysm, with one slice cut at the aneurysm and two slices cut at the non-aneurysm, and stained with HE; the non-stented segment was paraffin-embedded, with one slice cut at the proximal and one at the distal end; HE staining was performed for histopathological evaluation.

### Scanning Electron Microscope (SEM) Analysis

One animal each at 7D (7 days), 30D (30 days) and 60D (60 days) was randomly selected, and the aneurysm-carrying artery and abdominal aorta implanted at the above time points were collected for SEM to observe the endothelialization of the flow-directed stent at the aneurysm neck site and the opening site of the branch vessels at the abdominal aorta site.

Note: Only the abdominal aorta was examined by SEM at the 7D time point, the carrier artery SEM at the 30D time point was to be determined, and the carrier artery and abdominal aorta were examined by SEM at the 60D time point.

### Results

Checking of eG™ NTMA Film Grafting on the Electrografted Coupon

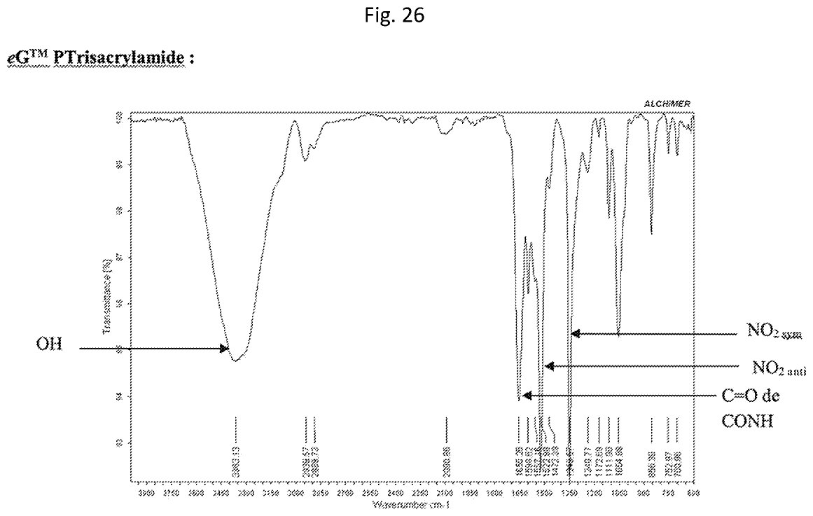

The infra-red spectrum of the eG™ NTMA film is presented in FIG. 26.

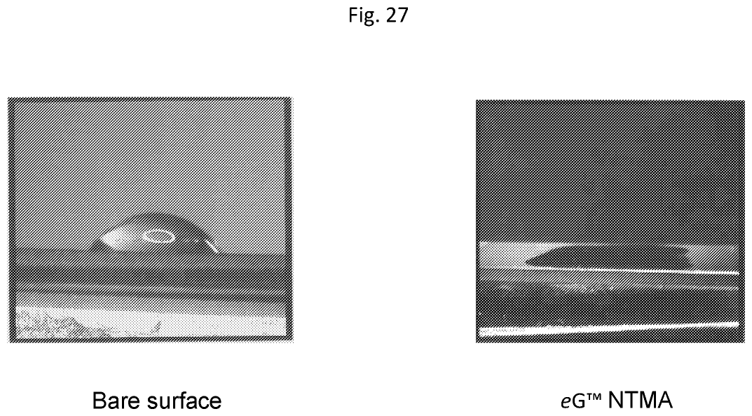

Moreover, a comparison of the hydrophilic character of the samples can be visualized by the shape of a water droplet (FIG. 27): on the bare surface, the water droplet stands on the surface, while on the eG™ NTMA surface, the droplet is totally flat, and the water contact angle is hardly measurable, showing that the surface is super-hydrophilic.

These results confirm efficient electrografting of the eG™ NTMA film on the comparative CFD stents.

### SEM Results

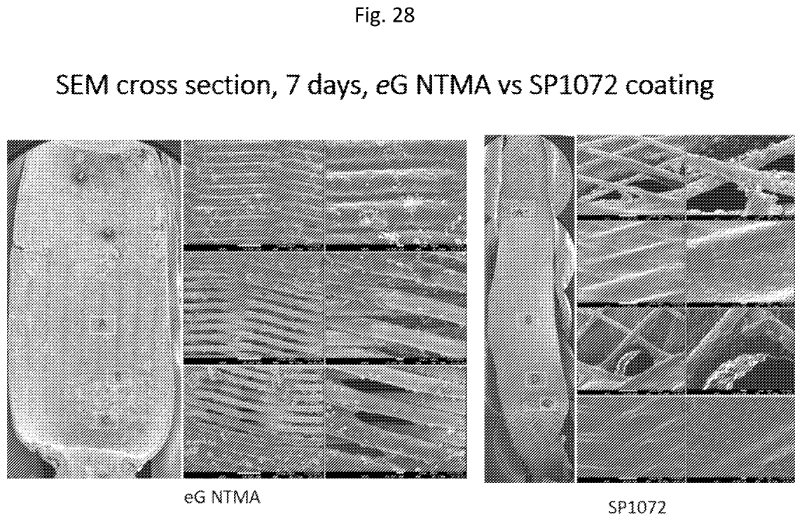

Exemplary SEM cross sections at day 7 of stents coated by eG™ NTMA or SP1072 peptide according to the invention are presented in FIG. 28.

For eG™ NTMA-coated stents (see left part), the stent was completely expanded in the vessel, with good stent vessel apposition and vessel lumen patency. Small amount of thrombus was scattered in partial area. For a small amount of stents, there was endothelial coverage on stent surface, and in areas without endothelial coverage, there were varying degrees of erythrocytes, inflammatory cells and platelet aggregation adhering to the stent surface. No blockage was found at collateral vessel caliber, indicating the patency of blood flow in collateral vessel.

For SP1072-coated stents (see right part), the stent was completely expanded in the vessel, with good stent vessel apposition and vessel lumen patency. No obvious thrombus was found, contrary to eG™ NTMA-coated stents. There was endothelial coverage on stent surface in partial areas (see notably the particularly good coverage in areas B and D of FIG. 26B), and in areas without endothelial coverage, there were small amounts of erythrocytes, inflammatory cells and platelet adhering to the stent surface. No blockage was found at collateral vessel caliber, indicating the patency of blood flow in collateral vessel.

### Histopathological Analysis

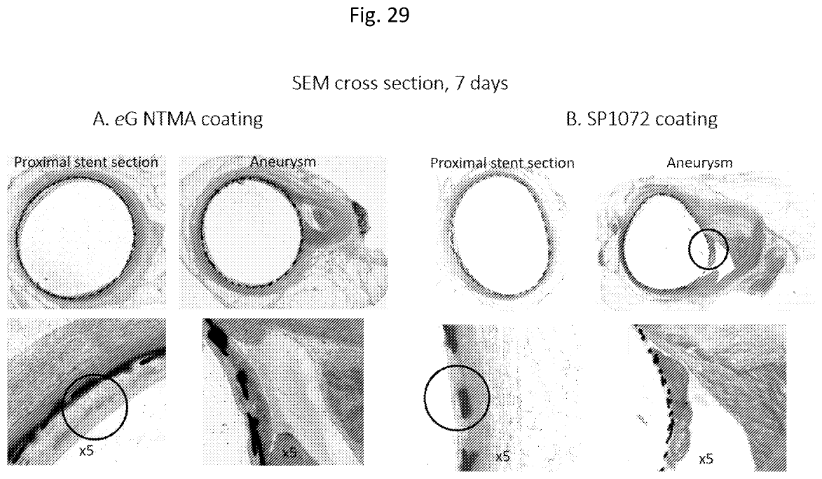

Exemplary pathology results at day 7 are presented in FIG. 29.

For eG™ NTMA-coated stents (see FIG. 29A), the stent in front of the aneurysmal neck (see right side) is covered by a neo-wall. The latter however appears infiltrated by leukocytes (black nuclei within the muscular portion, and in proximity of the stent struts, see right bottom picture), layered by a fresh fibrin layer on the inner side and packed red blood cells and platelets (dark grey areas in the fibrin layer, see black circle in the bottom left picture) on the outer side. Moreover, the thrombus in the aneurysmal sac does not appear organized (no evidence for the presence of polymerized extracellular matrix sheets) and fresh blood (red blood cells, platelets, leukocytes) is still entering in the thrombus. This is important because the fact that fresh blood can still enter and be trapped in the sac might increase the risk of late aneurysmal rupture because with the persistent arrival of fresh blood new waves of thrombosis/fibrinolysis occur and the enzymes of the two systems (thrombin, plasmin, etc., ) eventually drive the degradation of the aneurysmal wall. Altogether, these data indicate that the neo-tissue covering the neck of the aneurysm is hyperreactive (thrombotic and inflamed), possibly to the metal (foreign body), and leaky.

In contrast, for SP1072-coated stents (see FIG. 29A), the thrombus in the aneurysmal sac is “organized” because clear and aligned extracellular matrix sheets are visible across it, detectable as dark grey long and ondulated lines. Of note, the space between the neo-wall and the thrombus within the aneurysmal sac does not contain amorphous grey matter (fresh fibrin nor red blood cells), indicating that fresh blood is no longer allowed to enter and remain trapped there, hence reflecting a low risk of blood enzyme-driven aneurysmal wall degradation. The thick and regular shape of the arterial wall around the sac supports this assumption and the absence of amorphous material/dark nuclei within the thrombus support the assumption that it is impermeable to fresh blood platelets and coagulation factors.

Black circle in upper right picture: a neo-arterial wall, compact and rich in extracellular matrix (dark grey) and void of inflammation (no black nuclei) or thrombosis is being formed beneath the stent.

The presence of an organized thrombus on the outer side and of an organized neo-wall on the inner side support the fact that even if the blood may still enter in the large aneurysmal sac, as the one shown in the example, through the portions of the stent that are still patent, it cannot enter the thrombus nor clot over the neo-wall, allowing complete healing of the thrombus and occlusion in a shorter period as compared to situations where the blood could get in contact with a fresh thrombus and/or the naked stent. Indeed, once the cavity becomes virtual, the CD31 mimetic coating over the naked stent (portions devoid of a neo-wall) will favour the migration and growth of the adjacent endothelial cells on the device and in between, allowing to completely cover the organizing thrombus and hence occlude the aneurysmal neck.

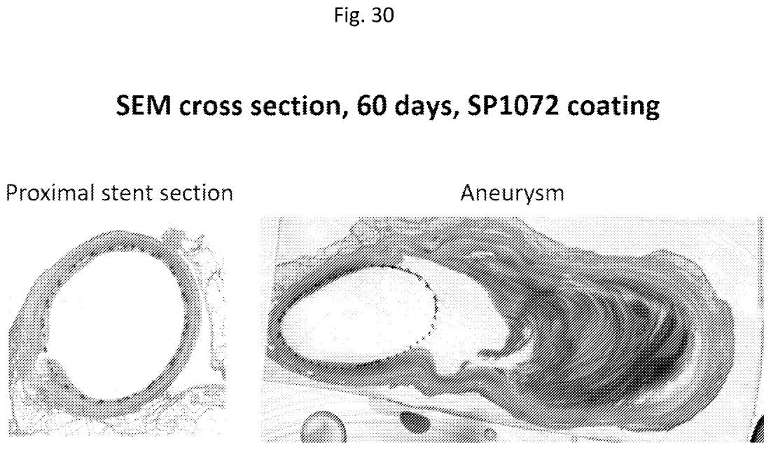

Exemplary pathology results at day 60 for the aneurysm of SP1072-coated stents are presented in FIG. 30. Despite the very large aneurysmal sac, the thrombus appears very well organized (long and ordered sheets of extracellular matrix throughout the thrombus, impermeable to fresh blood as detectable by the absence of leukocyte/platelet/RBC infiltration of the thrombus). As soon as the front of the organizing thrombus will reach the stent, the neck will be completely covered a neo-arterial wall. The latter will likely be well organized as suggested by the absence of inflammation/thrombosis of the arterial wall that is already in contact with the stent struts (absence of reaction to the foreign body).

### Conclusion

The above results show that the SP1072 coating:
promotes early endothelialization on the inner surface of the flow-diverter stents,
promotes the growth of an organized, blood-impermeable neo arterial wall at the entry of the aneurysm sac, allowing the building a well-organized and non-inflammatory thrombus inside the sac, and
At sites other than the aneurysm entry, endothelialization of the device is fast and complete without apparent inflammation nor neointima.

In view of the equivalent in vitro results obtained with other peptides according to the invention, similar in vivo results would also be expected by a skilled person.

### Example 4

### In Vivo Effect of Flow-Diverter Stents (CFD) Coated by Peptide SP1072 According to the Invention Compared to Stents Coated by Hydrophilic Polymer NTMA Only in the Absence of Administration of Anti-Platelets Compounds

The good results obtained in Example 3 suggest that stents coated with SP1072 (or another peptide according to the invention) might permit sufficiently rapid reendothelialization and sufficient absence of inflammation to prevent the need for administration of anti-platelets compounds, in particular anti-P2Y12 agents, after implantation.

A preliminary experiment testing this hypothesis was thus performed.

### Materials and Methods

The same materials and protocol as in Example 3 were used, except that:
Only 3 animals were used for each condition, and.
No aspirin nor clopidogrel was given to animals after the implantation.

### Results

Preliminary results show that:
animals implanted with SP1072-coated flow-diverter stents are still alive with no thrombus after 7 days (n=3),
in contrast, animal implanted with eG™ NTMA-coated stents were all dead by thrombosis at day 7 (n=3).

### Conclusion

While preliminary, this experiment suggests that following their implantation with medical devices coated with peptides according to the invention, the treatment of the patients might not require the use of anti-P2Y12, and even more generally of anti-platelet therapy (including aspirin).

This result is very important, as anti-platelets compounds and in particular anti-P2Y12 therapy cannot be used safely in individuals with hemorrhagic risk. Moreover, even in individuals not at risk of hemorrhage, anti-platelets compounds can have significant adverse effects (peptic ulcer with aspirin, neutropenia with anti-P2Y12 . . . ), which might be prevented by the use of medical devices coated with peptides according to the invention, if anti-platelets compounds are either not needed, or may be used at much lower concentration.

### Example 5

### In Vivo Effect of Self-Expendable Stents Coated by Peptide SP1072 According to the Invention Compared to Stents Coated by Hydrophilic Polymer NTMA Only

### Materials and Methods

### Stents

Self-expendable stents (ISS) are braided meshes made of nitinol, from Sinomed.

They are coated by one of the following coatings:
1. SP1072 peptide according to the invention, or
2. Hydrophilic polymer eG™ NTMA (obtained from the electrografting of N-[Tris(hydroxymethyl)Methyl] Acrylamide).

SP1072 peptide coating is performed as described in Example 3 above.

Hydrophilic polymer eG™ NTMA coated stents are obtained as described in Example 3 above.

### Animal Procedures

In this study, 5 healthy New Zealand white rabbits are selected as experimental animals, with 2 additional spare animals. There are 3 observation time points of 7, 14 and 28 days postoperatively, with 2 animals in the 7-day time point, 1 animal in the 14-day time point and 2 animals in the 28-day time point. A total of 4 stents are implanted in each experimental animal (2 each in test article A and test article B), and the abdominal aorta and bilateral iliac arteries are selected as the stent implantation sites in each animal, with 2 stents implanted in the abdominal aorta and 1 stent implanted in each of the bilateral iliac arteries. The postoperative observation period is followed up by imaging, and one sample from each animal in groups A and B is randomly selected for SEM, and the remaining samples are analyzed histopathologically. The general design of the experiment is as follows.

TABLE 3
General experimental design

| | | Number of | | | |
|---|---|---|---|---|---|
| | | stent implants | | | |
| Time | N. of | Test article | Test article | Implant | |
| Point | animals | group A | group B | location | Experiment content |
| 7 days | 1 | 2 | 2 | Abdominal | 1. adaptation period, |
| | | | | aorta with | preoperative, pre-taking blood |
| | | | | bilateral | routine and serum chemistry |
| 14 days | 1 | 2 | 2 | external | tests. |
| | | | | iliac | 2. daily observation of |
| | | | | arteries | experimental animals during the |
| 28 days | 1 | 2 | 2 | | postoperative observation |
| | | | | | feeding phase. |
| | | | | | 3. dissection and gross |
| | | | | | observation and photography. |
| | | | | | 4. electron microscopy scanning |
| | | | | | analysis. |
| | | | | | 5. target vessels for |
| | | | | | histopathological analysis. |

Note:
Test article group A and group B are two different coatings of self-expanding stents.

### Surgical Procedure:

(1) Anesthesia was performed as follows:

All surgical operations are performed using aseptic techniques, and the experimental animals are under general anesthesia during the implantation procedure.

On the day of surgery, the experimental animals are sedated and anesthesia is induced by intramuscular injection of 6 mg/kg of Sutex® 50. If needed, experimental animals can be anesthetized with isoflurane by inhalation using a breathing mask.

After successful induction of anesthesia, the experimental animal is connected to a ventilator device after being intubated through the oral plain view trachea to establish respiratory access and allow continuous inhalation of a mixture of anesthetics and oxygen to maintain anesthesia. It may be necessary to administer some atropine to the experimental animal to stop vomiting before the operation to prevent the animal from asphyxiation due to vomitus.

The anesthetized intubated experimental animal is placed on the operating table in the lateral or supine position, the animal is bound using a restraint band, and the position is photographed and recorded (to facilitate the same position and angle used for later observation). The surgical area of the right hind limb is prepared, disinfected, and sheeted. If the position of the animal is changed, the sheeting and preparation of the surgical site will need to be repeated.

An intravenous needle is also placed in one of the peripheral venous vessels and medications or rehydration fluids are administered through the catheter as needed.

(2) A 5F vascular sheath is inserted into the left or right common carotid artery to establish vascular access.

(3) A 5F catheter is introduced into the descending aorta of the heart via vascular access under the guidance of a guidewire, and iliac artery angiography is performed. Quantitative arterial vascular measurements are performed after angiography to guide the selection of the stent implantation site.

(4) Placement of microcatheter: intravenous heparin is treated with systemic heparinization, and the microcatheter is placed. The microcatheter is pushed through the microguide wire to the site of the iliac artery where the experimental animal stent is to be implanted, and the microguide wire is withdrawn.

(5) Iliac artery stent implantation: The system enters the microcatheter through the introducer sheath, the push rod is pushed forward to the appropriate position, the introducer sheath is withdrawn, the push rod continues to be pushed until the stent is pushed into the iliac artery, and the stent position is adjusted to ensure that the stent is in the intended position within the vessel. Secure the push rod and retract the microcatheter to begin partial release of the stent to the retrieval point position. If the position of the stent is satisfactory, continue to retract the microcatheter to release the stent completely; if the position is not satisfactory, push the microcatheter backwards to retrieve the stent into the microcatheter and adjust the position to release it again. After the stent placement is completed, withdraw the delivery system, push the micro-guide wire into the micro-catheter again, push the micro-guide wire together with the micro-catheter into the other side of the iliac artery, and complete the implantation of the stent on the other side in the same way.

(6) Stent implantation in the abdominal aorta: After the release of the iliac artery location test pin stent is completed, the microcatheter is retracted and placed in the location of the abdominal aorta, and two sets of stents are placed in the abdominal aorta in the same way. At the end of the procedure, all instruments and equipment are removed from the experimental animal.

(7) Wait for the animals to wake up and keep them normally until the end of the experiment.

In addition to heparin sodium injection 150 U/kg by IV during implantation, aspirin is given to the animals at 5 mg/kg by feeding once a day for 3 days before surgery and at 5 mg/kg by feeding postoperative to endpoint, and clopidogrel is given to the animals at 18.75 mg once a day by feeding for 3 days before surgery and at 18.75 mg by feeding postoperative to endpoint.

### Histopathological Analysis

The collected tissues of the stented segments of the carrier arteries are preserved in 10% neutral formalin for more than 48 hours of immersion fixation, dehydrated in alcohol gradient and treated with xylene transparency for histopathological analysis.

The resin-embedded stented segment of the iliac artery is sectioned transversely and stained with HE; the non-stented segment is paraffin-embedded, with one slice cut at the proximal and one at the distal end; HE staining is performed for histopathological evaluation.

### Scanning Electron Microscope (SEM) Analysis

One animal each at 7D (7 days), 14D (14 days) and 28D (28 days) is randomly selected, and the stented iliac artery and abdominal aorta implanted at the above time points are collected for SEM to observe the endothelialization of the ISS stent.

### Results

Based on results already obtained with CFD stents in Example 3, a good endothelialization with limited neointima formation is expected for the SP1072 peptide coated ISS stents, better than for the eG™ NTMA electrografted ISS stents.

### Example 6

### In Vivo Effect of Balloon Expandable CoCr Stents Coated by Peptide SP1072 According to the Invention Compared to Bare Metal Stents and HT Supreme Stents

### Materials and Methods

### Stents

Three types of balloon expandable stents are used:
CoCr stents coated by SP1072 peptide,
Bare metal stents, and
FT Supreme stent commercialized by Sinomed, which contains an eG BuMA primer layer and a 10 μm thick PLGA (poly(lactic-co-glycolic acid)) layer with a 1.2 μg/mm2 concentration of Sirolimus.

### Animal Procedures

In this study, 9 healthy New Zealand white rabbits are selected as experimental animals and 2 spare animals. The animals are divided into three observation time points: 7 days, 14 days and 28 days after surgery, with three animals at each time point. Each animal is implanted with 4 stents, and the abdominal aorta and bilateral iliac arteries of each animal are selected as the stent implantation sites. 2 stents are implanted in the abdominal aorta, and 1 stent is implanted in each bilateral iliac artery. At the end of the postoperative observation period, two samples of each animal with different coatings are randomly selected for SEM, and the rest of the samples are analyzed histopathologically. The general design of the experiment is shown in Table 4 below.

| | | Number | | |
|---|---|---|---|---|
| Time | Number of | of stent | Implant | |
| Point | animals | implants | location | Experiment content |
| 7 days | 3 | 12 | Abdominal | 1. routine blood and serum chemistry |
| | | | aorta with | testing during the adaptation period, |
| | | | bilateral | preoperatively, and before sampling; |
| 14 days | 3 | 12 | external | 2. daily observation of experimental |
| | | | iliac | animals during the postoperative |
| | | | arteries | observation and feeding stage; |
| 28 days | 3 | 12 | | 3. dissection and gross observation |
| | | | | and photography; |
| | | | | 4. electron microscopy scanning |
| Standby | 2 | 8 | | analysis; |
| (28 days) | | | | 5. histopathological analysis of |
| | | | | target vessels. |

### Surgical Procedures

(1) Anesthesia:

All surgical operations were performed using aseptic techniques, and the experimental animals were under general anesthesia during the implantation procedure.

On the day of surgery, the experimental animals are sedated and anesthesia is induced by intramuscular injection of 6 mg/kg of Sutex® 50. If needed, the experimental animals can be anesthetized with isoflurane by inhalation using a breathing mask.

After successful induction of anesthesia, the experimental animal is connected to a ventilator device after being intubated through the oral plain view trachea to establish respiratory access and allow continuous inhalation of a mixture of anesthetic and oxygen to maintain anesthesia. It may be necessary to administer some atropine to the experimental animal to stop vomiting before the operation to prevent the animal from asphyxiation due to vomitus.

The anesthetized and intubated experimental animal is placed on the operating table in the lateral or supine position and the animal is bound using a restraint band. The surgical area is prepared, disinfected, and sheeted. If the position of the animal is changed, the surgical site needs to be re-sheeted and prepared.

An intravenous needle is also placed in one of the peripheral venous vessels and medications or rehydration fluids are administered through the catheter as needed.

(2) A 6F vascular sheath is inserted into the left or right common carotid artery to establish vascular access.

(3) A 5F catheter was introduced into the descending aorta of the heart via vascular access under the guidance of a guidewire, and abdominal aortic and iliac artery angiography was performed. Quantitative arterial vascular measurements were performed after angiography to guide the selection of the stent implantation site.

(4) Iliac artery stent implantation: Withdraw the contrast catheter, prepare the stent delivery system, evacuate the air in the balloon of the delivery system until the negative pressure is reached, and then allow the balloon to inhale the contrast agent and heparinized saline mixture. The stent delivery system is guided by a 0.014 guidewire to deliver the stent to the selected stent implantation site. After the stent is delivered to the iliac artery vascular implantation site, the balloon dilatation pressure pump pressurizes the stent to open it and save the fluoroscopic image to record the stent implantation information. The balloon is dilated with the appropriate pressure at a ratio of 1.10-1.20:1 between the stent diameter and the target vessel diameter at the implantation site, and the ballast stent is released and held for 30 seconds to ensure good wall apposition of the implanted stent.

(5) After stent implantation on one side, the delivery balloon was withdrawn from the body and another angiogram was performed to evaluate the implanted stent. Stenting of the other side of the iliac artery was completed in the same way.

(6) Stent implantation in the abdominal aorta: After completion of stent release in the iliac artery location, 2 sets of stents were placed in the abdominal aorta in the same way. After the surgery, all instruments and equipment were removed from the experimental animals.

(7) Wait for the animals to wake up and keep them normally until the end of the experiment.

In addition to heparin sodium injection 150 U/kg by IV during implantation, antiplatelet therapy (aspirin and clopidogrel) was given to the animals at 5 mg/kg each by feeding once a day for 3 days before surgery and at 5 mg/kg each by feeding postoperative to endpoint.

### Histopathological Analysis

The collected tissues of the stented segments of the carrier arteries are preserved in 10% neutral formalin for more than 48 hours of immersion fixation, dehydrated in alcohol gradient and treated with xylene transparency for histopathological analysis.

The resin-embedded stented segment of the iliac artery is sectioned transversely and stained with HE; the non-stented segment is paraffin-embedded, with one slice cut at the proximal and one at the distal end; HE staining is performed for histopathological evaluation.

### Scanning Electron Microscope (SEM) Analysis

One animal each at 7D (7 days), 14D (14 days) and 28D (28 days) is randomly selected, and the stented iliac artery and abdominal aorta implanted at the above time points are collected for SEM to observe the endothelialization of the ISS stent.

### Results

Based on results already obtained with CFD stents in Example 3, a good endothelialization with limited neointima formation is expected for the SP1072 peptide coated balloon expandable stents, better than for the balloon expandable bare metal stents or HT Supreme stents.

From the above description, the advantages of the present invention will be clear to the person skilled in the art.

Whereas the P8RI sequence can uphold the clusterisation of truncated CD31 molecules expressed by activating cells, the presence of the invention peptides allows the engagement of intact CD31 molecules on all the healthy endothelial cells and resting blood platelets and leukocytes that can enter in contact with an implanted device.

Those cells can therefore receive the “leave-me-alone” signal delivered by the trans-homophilic engagement of CD31, which is essential to maintain the homeostasis in the circulation and vascularized tissues.

Thrombotic or life-threatening occurrence of hemorrhagic or thromboembolic complications have impaired the use of endovascular devices.

The devices bearing the mimicking peptides of the present invention are rapidly integrated, because they are perceived by blood platelets and leukocytes as a healthy endothelium, a “self” component.

Furthermore, their ability to be rapidly endothelialized with a physiologic endothelial cell phenotype also limits platelet and leukocyte activation at the site of device implantation in the long-term.

1. A medical device comprising a coating comprising a peptide mimicking the trans-homophilic CD31-CD31 domain 1 and 2 intercellular interaction.

2. The medical device of claim 1, wherein the peptide mimicking the trans-homophilic CD31-CD31 domain 1 and 2 intercellular interaction comprises one of the following sequences:
a)

| SEQ. ID no. 53 | |
|---|---|
| | QHXXLFYKDDXXFYNISSXX |

wherein:
1Q may be any one of: Q, C, L, K, or R,
2H may be any one of: H, I, V, Q or R,
5L may be any one of: L, R, V, F or E,
9D may be any one of: D, E or N,
13F may be any one of: F, V, L or I,
14Y may be any one of: Y, H, R or N,
15N may be any one of: N or D,
16I may be any one of: I, V, T or A,
17S may be any one of: S or T,
18S may be any one of: S or T,
independently one from another, wherein the other amino acids X may be any other amino acid.
b)

| SEQ. ID no. 54 | |
|---|---|
| | YKSTVIXNNKEKTTXE |

wherein:
2K may be any one of: K or R
3S may be any one of: C or S
4T may be any one of: T, R or S
5V may be any one of: V or A
6I may be any one of: I, K, V, L, T or S
8N may be any one of: N, S or D
9N may be any one of: N, S, K or R
11E may be any one of: E, Q, V, K or M
12K may be any one of: K or R
13T may be any one of: T, A or P
14T may be any one of: T or S
16E may be any one of: E, A, Q or D
independently one from another, wherein the other amino acids X may be any other amino acid.
c)

| SEQ. ID no. 55 | |
|---|---|
| | PXCTLDKKEXIQGGXVXVNCSVPEEK |

wherein:
3C may be any one of: C, V, M or I
4T may be any one of: T, I, M or E
5L may be any one of: L or V
6D may be any one of: D or N
7K may be any one of: K or R
8K may be any one of: K, T, M, R or I
11I may be any one of: I, T, M, V or E
12Q may be any one of: Q or E
14G may be any one of: G or E
16V may be any one of: V or I
18V may be any one of: V or I
19N may be any one of: N, T, R, S, G or H
22V may be any one of: V, M or L
23P may be any one of: P, Q, K, E, L or R
24E may be any one of: E, G or N
26K may be any one of: K, Q, E, R or N
independently one from another, wherein the other amino acids X may be any other amino acid; or
d) any combination of a) to c).

3. The medical device of claim 1, wherein the peptide mimicking the trans-homophilic CD31-CD31 domain 1 and 2 intercellular interaction comprises one of the following sequences:

| | Seq. ID. |
|---|---|
| Amino acidic sequence | No. |
| HQMLFYKDDVLFYNISS | 4 |
| YKSTVIVNNKEKTTAE | 5 |
| PRVTLDKKEAIQGGIVRVNSSVPEEK | 6 |
| HQMLFYKDDVLFYNISSC | 7 |
| SHQMLFYKDDVLFYNISSS | 8 |
| QHQMLFYKDDVLFYNISSMK | 9 |
| CHQMLFYKDDVLFYNISSCK | 10 |
| YKDDVLFYNISSMKST | 11 |
| PQHQMLFYKDDVLFYNISSMK | 12 |
| FADVSTTSHVKPQHQMLFYKDDVLFYNISSMKSTESYFI | 13 |
| PEVRIYDSGTYK | |
| PQHQMLFYKDDVLFYNISSMKSTESYFIPEVRIYDSGTY | 14 |
| KSTVIV | |
| YKDDVLFYNISSMKSTESYFIPEVRIYDSGTYKSTVIV | 15 |
| HQMLFYKDDVLFYNISSMKSTESYFIPEVRIYDSGTYKS | 16 |
| TVIV | |
| LFYKDDVLFYNISSMKSTESYF | 17 |
| PQHQMLFYKDDVLFYNISSMK | 18 |
| QHQMLFYhCDDVLFYNISSMK, being | 28 |
| hC = homocysteine | |
| YKSTVIVNNKEKTTAE | 19 |
| DDVLFYNISSMK | 20 |
| QHQMLFY | 21 |
| CYKSTVIVNNKEKTTAEYC | 22 |
| hCYKSTVIVNNKEKTTAEYhC, being | 23 |
| hC = homocysteine | |
| GTYKSTVIVNNKEKTTAEYQ | 24 |
| PRCTLDKKEAIQGGIVRVNCSVPEEK | 25 |
| RDQNFVILEFP | 26 |

4. The medical device according to claim 2, wherein the peptide mimicking the trans-homophilic CD31-CD31 domain 1 and 2 intercellular interaction comprises two of said sequences.

5. The medical device of claim 1, wherein the peptide mimicking the trans-homophilic CD31-CD31 domain 1 and 2 intercellular interaction comprises:
a peptide based on structure (IA):

| | Seq. ID |
|---|---|
| Aminoacidic sequence (IA) | No. |
| HQMLFYKDDVLFYNISSC | 7 |
| SHQMLFYKDDVLFYNISSS | 8 |
| QHQMLFYKDDVLFYNISSMK | 9 |
| CHQMLFYKDDVLFYNISSCK | 10 |
| YKDDVLFYNISSMKST | 11 |
| PQHQMLFYKDDVLFYNISSMK | 12 |
| FADVSTTSHVKPQHQMLFYKDDVLFYNISSMKSTESYFIP | 13 |
| EVRIYDSGTYK | |
| PQHQMLFYKDDVLFYNISSMKSTESYFIPEVRIYDSGTYK | 14 |
| STVIV | |
| YKDDVLFYNISSMKSTESYFIPEVRIYDSGTYKSTVIV | 15 |
| HQMLFYKDDVLFYNISSMKSTESYFIPEVRIYDSGTYKST | 16 |
| VIV | |
| LFYKDDVLFYNISSMKSTESYF | 17 |
| PQHQMLFYKDDVLFYNISSMK | 18 |
| QHQMLFYhCDDVLFYNISSMK | 28 |

hC = homocysteine
and a peptide based on structure (IB):

| Aminoacidic sequence (IB) | Seq. ID No. |
|---|---|
| YKSTVIVNNKEKTTAE | 19 |
| DDVLFYNISSMK | 20 |
| QHQMLFY | 21 |
| CYKSTVIVNNKEKTTAEYC | 22 |
| hCYKSTVIVNNKEKTTAEYhC | 23 |
| GTYKSTVIVNNKEKTTAEYQ | 24 |

hC = homocysteine
or
a peptide based on structure (IA):

| | Seq. ID |
|---|---|
| Aminoacidic sequence (IA) | No. |
| HQMLFYKDDVLFYNISSC | 7 |
| SHQMLFYKDDVLFYNISSS | 8 |
| QHQMLFYKDDVLFYNISSMK | 9 |
| CHQMLFYKDDVLFYNISSCK | 10 |
| YKDDVLFYNISSMKST | 11 |
| PQHQMLFYKDDVLFYNISSMK | 12 |
| FADVSTTSHVKPQHQMLFYKDDVLFYNISSMKSTESYFIP | 13 |
| EVRIYDSGTYK | |
| PQHQMLFYKDDVLFYNISSMKSTESYFIPEVRIYDSGTYK | 14 |
| STVIV | |
| YKDDVLFYNISSMKSTESYFIPEVRIYDSGTYKSTVIV | 15 |
| HQMLFYKDDVLFYNISSMKSTESYFIPEVRIYDSGTYKST | 16 |
| VIV | |
| LFYKDDVLFYNISSMKSTESYF | 17 |
| PQHQMLFYKDDVLFYNISSMK | 18 |
| QHQMLFYhCDDVLFYNISSMK | 28 |

hC = homocysteine
and a peptide based on structure (IC):

| Aminoacidic sequence (IC) | Seq. ID No. |
|---|---|
| PRCTLDKKEAIQGGIVRVNCSVPEEK | 25 |

6. The medical device according to claim 5, wherein said peptide mimicking the trans-homophilic CD31-CD31 domain 1 and 2 intercellular interaction is characterized by comprising the followings sequences:

| Aminoacidic sequences | Seq. ID No. |
|---|---|
| GTYKSTVIVNNKEKTTAEYQ and | 24 and 28 |
| QHQMLFYhCDDVLFYNISSMK | (SEQ ID No. |
| | 37 and 38) |
| PRVTLDKKEAIQGGIVRVNSSVPE | 6 and 28 |
| EK and QHQMLFYhCDDVLFYNI | (SEQ ID No. |
| SSMK | 39 and 40) |

7. The medical device according to claim 1, wherein said peptide mimicking the trans-homophilic CD31-CD31 domain 1 and 2 intercellular is a cyclic peptide.

8. The medical device according to claim 1, wherein said peptide mimicking the trans-homophilic CD31-CD31 domain 1 and 2 intercellular comprises a linker and/or a spacer and/or a tail at any terminus or linked to an aminoacidic residue.

9. The medical device according to claim 8, wherein said linker and/or a spacer and/or a tail is represented by:

| SEQ. ID No. 27 | |
|---|---|
| | GGSGGSGG |
| | one or more PEG4 units |
| | one or more Ttds units |

or combinations thereof.

10. The medical device according to claim 1, wherein said peptide mimicking the trans-homophilic CD31-CD31 domain 1 and 2 intercellular comprises a modification at the C-terminus and/or at the N-terminus.

11. The medical device according to claim 10, wherein said modification is selected from:
Ac—
Br—CH₂—CO—
CH₂—CO—
CH₂—CONH₂—
Ac—K(N₃)
—NH₂
—CO—NH₂

12. The medical device according to claim 1, wherein said peptide mimicking the trans-homophilic CD31-CD31 domain 1 and 2 intercellular comprises any one of the following modifications:
a cysteine residue may be substituted by the corresponding homocysteine,
an L-amino acid residue may be substituted by the corresponding D-amino acid residue.

13. The medical device according to claim 1, wherein said peptide mimicking the trans-homophilic CD31-CD31 domain 1 and 2 intercellular is characterized by having one of the following structures:

| | SEQ. |
|---|---|
| | ID |
| Aminoacidic sequence | No. |
| QHQMLFYCDDVLFYNISSMK-CONH₂ | 1 |
| (BrCH₂CO-GTYKSTVIVNNKEKTTAEYQ-(Ttds)₄-K(N₃)-NH₂) | 2 |
| Ac-K(N₃)-(Ttds)₄-PRVTLDKKEAIQGGIVRVNSSVPEEK-Ttds-K(BrCH₂CO)- | 3 |
| CONH₂ | |
| Ac-c*HQMLFYKDDVLFYNISSC*-GGSGGSGG-K(N₃)-CONH₂ | 29 |
| SHQMLFYKDDVLFYNISSS-GGSGGSGG-K(N₃)-CONH₂ | 30 |
| Ac-KKKc*HQMLFYKDDVLFYNISSC*-Ttds-Ttds-K(N₃)-CONH₂ | 31 |
| QHQMLFYKDDVLFYNISSMK-Ttds-Ttds-Ttds-Ttds-K(N₃)-CONH₂ | 32 |
| hCHQMLFYKDDVLFYNISShCK-Ttds-Ttds-Ttds-Ttds-K(N₃)-CONH₂ | 33 |
| YKSTVIVNNKEKTTAE-PEG₄-K(N₃)-CONH₂ | 34 |
| Ac-K(N₃)-PEG₄-PRVTLDKKEAIQGGIVRVNSSVPEEK-CONH₂ | 35 |
| Ac-K(N₃)-(Ttds)₄-PRhCTLDKKEAIQGGIVRVNhCSVPEEK-CONH₂ | 36 |
| [^{#}CH₂CONH₂GTYKSTVIVNNKEKTTAEYQ-(Ttds)₄-K(N₃)-NH₂]- | 37 |
| [QHQMLFYhC*DDVLFYNISSMK-CONH₂] | and |
| | 38 |
| [Ac-K(N₃)-(Ttds)₄-PRVTLDKKEAIQGGIVRVNSSVPEEK-Ttds-K^{#}(CH₂CO)- | 39 |
| CONH₂]-[QHQMLFYhC^{#}DDVLFYNISSMK-CONH₂] | and |
| | 40 |
| Ac-K(N₃)-GGSGGSGG-YKDDVLFYNISSMKST-NH₂ | 41 |
| Ac-YKDDVLFYNISSMKST-GGSGGSGG-K(N₃)-NH₂ | 42 |
| Ac-PQHQMLFYKDDVLFYNISSMK(GGSGGSGG-K(N₃))STESYFI-CONH₂ | 43 |
| Ac-K(N₃)-GGSGGSGG-FADVSTTSHVKPQHQMLFYKDDVLFYNISSMKSTESYFIPE | 44 |
| VRIYDSGTYK-CONH₂ | |
| Ac-K(N₃)-GGSGGSGG-PQHQMLFYKDDVLFYNISSMKSTESYFIPEVRIYDSGTYKS | 45 |
| TVIV-CONH₂ | |
| Ac-K(N₃)-GGSGGSGG-YKDDVLFYNISSMKSTESYFIPEVRIYDSGTYKSTVIV- | 46 |
| CONH₂ | |
| Ac-K(N₃)-GGSGGSGG-HQMLFYKDDVLFYNISSMKSTESYFIPEVRIYDSGTYKSTV | 47 |
| IV-CONH₂ | |
| Ac-LFYKDDVLFYNISSMKSTESYF-GGSGGSGG-K(N₃)-CONH₂ | 48 |
| Ac-LFYKDDVLFYNISSMKSTESYF-Ttds-Ttds-K(N₃)-CONH₂ | 49 |
| Ac-PQHQMLFYKDDVLFYNISSMK(GGSGGSGG-K(N₃))STESYFIKK-CONH₂ | 50 |
| RDQNFVILEFP-PEG₄-K(N₃)-NH₂ | 51 |

wherein
hC: homoCysteine;
c: D-Cysteine;
**position for disulfide bridge formation of cyclic peptides
^{#}acetyl thioether linkage

14. (canceled)

15. (canceled)

16. The method according to claim 21, wherein the vascular pathology is selected from the group comprising: heart valve pathology, atherosclerosis, thrombosis, ischemia, hemorrhage, restenosis, aneurism.

17. (canceled)

18. (canceled)

19. The medical device according to claim 1, which is partially or fully coated with the coating comprising a peptide mimicking the trans-homophilic CD31-CD31 domain 1 and 2 intercellular interaction.

20. The medical device according to claim 19, which is selected in the group comprising: balloon expandable stents, self-expandable scaffolds, the polymer tube of graft-stents, flow diverting meshes, aortic tubes, cardiac valves, stent retriever, transcatheter mitral valve device, catheter, leaflet or any portions thereof.

21. A method for the prevention or for the treatment of heart and vascular pathologies or for the prevention of a condition represented by the in-stent stenosis, comprising the use of a device according to claim 1.

22. (canceled)

23. (canceled)

24. The method according to claim 21, which comprises the implantation of a medical device comprising a coating comprising a peptide mimicking the trans-homophilic CD31-CD31 domain 1 and 2 intercellular interact.

25. The method according to claim 24, wherein after implantation the individual into which the medical device is implanted:
a) does not take anti-P2Y12 therapy;
b) takes a significantly lower dose of anti-P2Y12 therapy than recommended for drug-eluting stents within a traditional dual anti-platelet therapy (DAPT);
c) takes anti-P2Y12 therapy during a significantly shorter timeframe than recommended for drug-eluting stents within a traditional dual anti-platelet therapy (DAPT); or
d) any combination of b) and c).

26. The method according to claim 25, wherein the individual into which the medical device is implanted suffers from a bleeding disorder.


SEQUENCE LISTING (55 sequences)

SEQ ID NO: 1 - protein, 20 aa, synthetic construct
QHQMLFYCDDVLFYNISSMK

SEQ ID NO: 2 - protein, 20 aa, synthetic construct
  BINDING 20: note = (Ttds)4-K(N3)
GTYKSTVIVNNKEKTTAEYQ

SEQ ID NO: 3 - protein, 26 aa, synthetic construct
  BINDING 1: note = K(N3)-(Ttds)4
  BINDING 26: note = Ttds-K-Br-CH2CO
PRVTLDKKEAIQGGIVRVNSSVPEEK

SEQ ID NO: 4 - protein, 17 aa, synthetic construct
HQMLFYKDDVLFYNISS

SEQ ID NO: 5 - protein, 16 aa, synthetic construct
YKSTVIVNNKEKTTAE

SEQ ID NO: 6 - protein, 26 aa, synthetic construct
PRVTLDKKEAIQGGIVRVNSSVPEEK

SEQ ID NO: 7 - protein, 18 aa, synthetic construct
HQMLFYKDDVLFYNISSC

SEQ ID NO: 8 - protein, 19 aa, synthetic construct
SHQMLFYKDDVLFYNISSS

SEQ ID NO: 9 - protein, 20 aa, synthetic construct
QHQMLFYKDDVLFYNISSMK

SEQ ID NO: 10 - protein, 20 aa, synthetic construct
CHQMLFYKDDVLFYNISSCK

SEQ ID NO: 11 - protein, 16 aa, synthetic construct
YKDDVLFYNISSMKST

SEQ ID NO: 12 - protein, 21 aa, synthetic construct
PQHQMLFYKDDVLFYNISSMK

SEQ ID NO: 13 - protein, 51 aa, synthetic construct
FADVSTTSHVKPQHQMLFYKDDVLFYNISSMKSTESYFIPEVRIYDSGTYK

SEQ ID NO: 14 - protein, 45 aa, synthetic construct
PQHQMLFYKDDVLFYNISSMKSTESYFIPEVRIYDSGTYKSTVIV

SEQ ID NO: 15 - protein, 38 aa, synthetic construct
YKDDVLFYNISSMKSTESYFIPEVRIYDSGTYKSTVIV

SEQ ID NO: 16 - protein, 43 aa, synthetic construct
HQMLFYKDDVLFYNISSMKSTESYFIPEVRIYDSGTYKSTVIV

SEQ ID NO: 17 - protein, 22 aa, synthetic construct
LFYKDDVLFYNISSMKSTESYF

SEQ ID NO: 18 - protein, 21 aa, synthetic construct
PQHQMLFYKDDVLFYNISSMK

SEQ ID NO: 19 - protein, 16 aa, synthetic construct
YKSTVIVNNKEKTTAE

SEQ ID NO: 20 - protein, 12 aa, synthetic construct
DDVLFYNISSMK

SEQ ID NO: 21 - protein, 7 aa, synthetic construct
QHQMLFY

SEQ ID NO: 22 - protein, 19 aa, synthetic construct
CYKSTVIVNNKEKTTAEYC

SEQ ID NO: 23 - protein, 19 aa, synthetic construct
  SITE 1: note = X is homocysteine
  SITE 19: note = X is homocysteine
  DISULFID 1..19: note = disulfide bond between homocysteine in positions 1 and 19
XYKSTVIVNNKEKTTAEYX

SEQ ID NO: 24 - protein, 20 aa, synthetic construct
GTYKSTVIVNNKEKTTAEYQ

SEQ ID NO: 25 - protein, 26 aa, synthetic construct
PRCTLDKKEAIQGGIVRVNCSVPEEK

SEQ ID NO: 26 - protein, 11 aa, synthetic construct
RDQNFVILEFP

SEQ ID NO: 27 - protein, 8 aa, synthetic construct
GGSGGSGG

SEQ ID NO: 28 - protein, 20 aa, synthetic construct
  SITE 8: note = X is homocysteine
QHQMLFYXDDVLFYNISSMK

SEQ ID NO: 29 - protein, 19 aa, synthetic construct
  DISULFID 1..19: note = disulfide bond
  SITE 1: note = D-amino acid
  BINDING 19: note = GGSGGSGG-K(N3)
  SITE 1: note = acetylated
CHQMLFYKDDVLFYNISSC

SEQ ID NO: 30 - protein, 19 aa, synthetic construct
  BINDING 19: note = GGSGGSGG-K(N3)
SHQMLFYKDDVLFYNISSS

SEQ ID NO: 31 - protein, 22 aa, synthetic construct
  SITE 4: note = D-amino acid
  DISULFID 4..22: note = disulfide bond
  BINDING 22: note = (Ttds)2-K(N3)
  SITE 1: note = acetylated
KKKCHQMLFYKDDVLFYNISSC

SEQ ID NO: 32 - protein, 20 aa, synthetic construct
  BINDING 20: note = -(Ttds)4-K(N3)
QHQMLFYKDDVLFYNISSMK

SEQ ID NO: 33 - protein, 20 aa, synthetic construct
  SITE 1: note = X is homocysteine
  SITE 19: note = X is homocysteine
  BINDING 20: note = -(Ttds)4-K(N3)
XHQMLFYKDDVLFYNISSXK

SEQ ID NO: 34 - protein, 16 aa, synthetic construct
  BINDING 16: note = PEG4-K(N3)
YKSTVIVNNKEKTTAE

SEQ ID NO: 35 - protein, 26 aa, synthetic construct
  BINDING 1: note = Ac-K(N3)-PEG4-
PRVTLDKKEAIQGGIVRVNSSVPEEK

SEQ ID NO: 36 - protein, 26 aa, synthetic construct
  BINDING 1: note = Ac-K(N3)-(Ttds)4
  SITE 3: note = X is homocysteine
  SITE 20: note = X is homocysteine
  DISULFID 3..20: note = disulfide bond
PRXTLDKKEAIQGGIVRVNXSVPEEK

SEQ ID NO: 37 - protein, 20 aa, synthetic construct
  SITE 1: note = glycine is linked to acetamide which is a site for acetylthioether linkage to aspartic acid at position 9 of SEQ. ID No. 38
  BINDING 20: note = -(Ttds)4-K(N3)
GTYKSTVIVNNKEKTTAEYQ

SEQ ID NO: 38 - protein, 20 aa, synthetic construct
  SITE 8: note = X is homocysteine
  CROSSLNK 9: note = acetyl thioether linkage to acetamido group of glycine in position 1 of SEQ. ID no. 37
QHQMLFYXDDVLFYNISSMK

SEQ ID NO: 39 - protein, 26 aa, synthetic construct
  BINDING 1: note = K(N3)-(Ttds)4
  BINDING 26: note = (Ttds)-K(CH2CO)-CONH2 wherein K is a site for acetyl thioether linkage to aspartic acid at position 9 of SEQ. ID no. 40
PRVTLDKKEAIQGGIVRVNSSVPEEK

SEQ ID NO: 40 - protein, 20 aa, synthetic construct
  SITE 8: note = X is homocysteine
  CROSSLNK 9: note = acetyl thioether linkage to acetamido group of lysine in position 26 of SEQ. ID no. 39
QHQMLFYXDDVLFYNISSMK

SEQ ID NO: 41 - protein, 16 aa, synthetic construct
  BINDING 1: note = K(N3)-GGSGGSGG
YKDDVLFYNISSMKST

SEQ ID NO: 42 - protein, 16 aa, synthetic construct
  BINDING 16: note = GGSGGSGG-K(N3)
YKDDVLFYNISSMKST

SEQ ID NO: 43 - protein, 28 aa, synthetic construct
  BINDING 21: note = GGSGGSGG-K(N3)
PQHQMLFYKDDVLFYNISSMKSTESYFI

SEQ ID NO: 44 - protein, 51 aa, synthetic construct; identical to SEQ ID NO: 13
  BINDING 1: note = K(N3)-GGSGGSGG

SEQ ID NO: 45 - protein, 45 aa, synthetic construct; identical to SEQ ID NO: 14
  BINDING 1: note = K(N3)-GGSGGSGG

SEQ ID NO: 46 - protein, 38 aa, synthetic construct; identical to SEQ ID NO: 15
  BINDING 1: note = K(N3)-GGSGGSGG

SEQ ID NO: 47 - protein, 43 aa, synthetic construct; identical to SEQ ID NO: 16
  BINDING 1: note = K(N3)-GGSGGSGG

SEQ ID NO: 48 - protein, 22 aa, synthetic construct
  BINDING 22: note = GGSGGSGG-K(N3)
LFYKDDVLFYNISSMKSTESYF

SEQ ID NO: 49 - protein, 22 aa, synthetic construct
  BINDING 22: note = Ttds-Ttds-K(N3)
LFYKDDVLFYNISSMKSTESYF

SEQ ID NO: 50 - protein, 30 aa, synthetic construct
  BINDING 21: note = GGSGGSGG-K(N3)
PQHQMLFYKDDVLFYNISSMKSTESYFIKK

SEQ ID NO: 51 - protein, 11 aa, synthetic construct
  BINDING 11: note = PEG4-K(N3)
RDQNFVILEFP

SEQ ID NO: 52 - protein, 8 aa, synthetic construct
KWPALFVR

SEQ ID NO: 53 - protein, 20 aa, synthetic construct
  VARIANT 1: note = Q may be substituted by any one of C or L or K or R
  VARIANT 2: note = H may be substituted by I or V or Q or R
  VARIANT 5: note = L may be substituted by R or V or F or E
  VARIANT 9: note = D may be substituted by E or N
  VARIANT 13: note = F may be substituted by V or L or I
  VARIANT 14: note = Y may be substituted by H or R or N
  VARIANT 15: note = N may be substituted by D
  VARIANT 16: note = I may be substittued by V or T or A
  VARIANT 17: note = S may be substituted by T
  VARIANT 18: note = S may be substituted by T
  VARIANT 3: note = X may be any amino acid
  VARIANT 4: note = X may be any amino acid
QHXXLFYKDDXXFYNISSXX

SEQ ID NO: 54 - protein, 16 aa, synthetic construct
  VARIANT 2: note = K may be substituted by R
  VARIANT 3: note = S may be substituted by C
  VARIANT 4: note = T may be substituted by R or S
  VARIANT 5: note = V may be substituted by A
  VARIANT 6: note = I may be substittued by K or V or L or T or S
  VARIANT 8: note = N may be substituted by S or D
  VARIANT 9: note = N may be substituted by S or K or R
  VARIANT 11: note = E may be substituted by Q or V or K or M
  VARIANT 12: note = K may be substituted by R
  VARIANT 13: note = T may besubstituted by A or P
  VARIANT 14: note = T may be substituted by S
  VARIANT 16: note = E may be substituted by A or Q or D
  VARIANT 7: note = X may be any amino acid
  VARIANT 15: note = X may be any amino acid
YKSTVIXNNKEKTTXE

SEQ ID NO: 55 - protein, 26 aa, synthetic construct
  VARIANT 2: note = X may be any amino acid
  VARIANT 10: note = X may be any amino acid
  VARIANT 15: note = X may be any amino acid
  VARIANT 17: note = X may be any amino acid
  VARIANT 3: note = V may be substituted by C or M or I
  VARIANT 4: note = T may be substituted by I or M or E
  VARIANT 5: note = L may be substituted by V
  VARIANT 6: note = D may be sustituted by N
  VARIANT 7: note = K may be susbstituted by R
  VARIANT 8: note = K may be substituted by T or M or R or I
  VARIANT 11: note = I may be substituted by T or M or V or E
  VARIANT 12: note = Q may be substituted by E
  VARIANT 14: note = G may be substituted by E
  VARIANT 16: note = V may be substituted by I
  VARIANT 18: note = V may be substituted by I
  VARIANT 19: note = N may be substituted by T or R or S or G or H
  VARIANT 22: note = V may be subsituted by M or L
  VARIANT 23: note = P may be substituted by Q or K or E or L or R
  VARIANT 24: note = E may be substituted by G or N
  VARIANT 26: note = K may be substituted by Q or E or R or N
PXCTLDKKEXIQGGXVXVNCSVPEEK